(19) United States
(12) Patent Application Publication
(10) Pub. No.: US 2022/0227798 A1
(43) Pub. Date: Jul. 21, 2022

(54) MANUFACTURE METHOD FOR AQUEOUS FORMULATION OF MANGANESE-CONTAINING COORDINATION COMPLEX, FORMULATION, AND METHOD OF TREATMENT

(71) Applicant: Galera Labs, LLC, Malvern, PA (US)

(72) Inventors: Robert A. BEARDSLEY, University City, MO (US); Jeffrey A. SCHOLTEN, Chesterfield, MO (US); James BLACKLEDGE, St. Louis, MO (US); Dennis P. RILEY, Chesterfield, MO (US); Otto F. SCHALL, Creve Coeur, MO (US); Robert MCKEAN, Chester Springs, PA (US)

(21) Appl. No.: 17/715,297

(22) Filed: Apr. 7, 2022

Related U.S. Application Data
(63) Continuation of application No. PCT/US20/55221, filed on Oct. 12, 2020.
(60) Provisional application No. 62/913,704, filed on Oct. 10, 2019.

(51) Int. Cl. C07F 13/00

(52) U.S. Cl. CPC C07F 13/00

(57) ABSTRACT

A method is provided for manufacturing an aqueous formulation of a manganese-containing coordination complex, by combining a source of the manganese-containing coordination complex with a source of chloride anion in an aqueous solution, and simultaneously with or following combination of the source of chloride anion and the source of manganese-containing coordination complex in the aqueous solution, providing a source of a dianion to the aqueous solution to form the aqueous formulation.

### CROSS-REFERENCE TO RELATED APPLICATIONS

The present application is a continuation of PCT Application Serial No. PCT/US2020/055221, filed Oct. 12, 2020, which claims priority to U.S. Provisional Application No. 62/913,704, filed on Oct. 10, 2019, both of which are hereby incorporated by reference herein in their entireties.

The present disclosure generally relates to a method of manufacture of an aqueous formulation of a manganese-containing coordination complex, such as a manganese-containing pentaaza macrocyclic ring complex, as well as formulations and methods of treatment therewith.

Aqueous formulations of manganese-containing coordination complexes may be prepared for a variety of different uses, including for use as parenteral drug formulations for the treatment of disease states. Parenteral formulations are typically required to have certain properties in order to be suitable for administration, such as a physiologically acceptable pH, the ability to maintain solubility of the compound being parenterally administered substantially without degradation of the formulation, and acceptable isotonicity. Aqueous formulations for parenteral administration generally also must be sterile, preferably contain no visibly discernible particles, and limited amounts of particles that are sized below the visible threshold.

Examples of manganese-containing coordination complexes that may be administered for treatment in aqueous formulations include manganese-containing pentaaza macrocyclic ring complexes having the macrocyclic ring system corresponding to Formula A, which have been shown to be effective in a number of animal and cell models of human disease, as well as in treatment of conditions afflicting human patients.

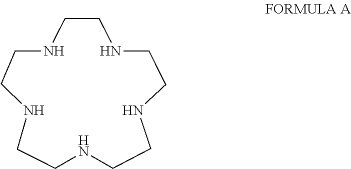 [SMILES: C1CNCCNCCNCCNCCN1]

For example, in a rodent model of colitis, one such compound, GC4403, has been reported to very significantly reduce the injury to the colon of rats subjected to an experimental model of colitis (see Cuzzocrea et al., Europ. J. Pharmacol., 432, 79-89 (2001)).

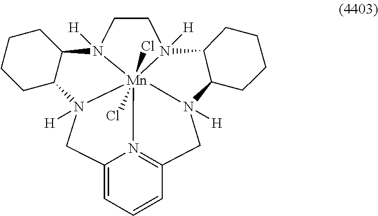

GC4403 has also been reported to attenuate the radiation damage arising both in a clinically relevant hamster model of acute, radiation-induced oral mucositis (Murphy et al., Clin. Can. Res., 14(13), 4292 (2008)), and lethal total body irradiation of adult mice (Thompson et al., Free Radical Res., 44(5), 529-40 (2010)). Similarly, another such compound, GC4419, has been shown to attenuate VEGFr inhibitor-induced pulmonary disease in a rat model (Tuder, et al., Am. J. Respir. Cell Mol. Biol., 29, 88-97 (2003)). Additionally, another such compound, GC4401 has been shown to provide protective effects in animal models of septic shock (S. Cuzzocrea, et.al., Crit. Care Med., 32(1), 157 (2004) and pancreatitis (S. Cuzzocrea, et.al., Shock, 22(3), 254-61 (2004)).

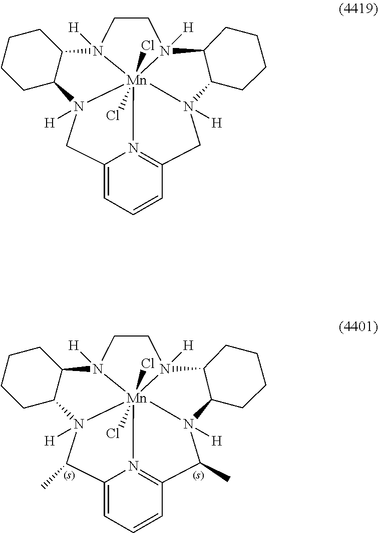

Certain of these compounds have also been shown to possess potent anti-inflammatory activity and prevent oxidative damage in vivo. For example, GC4403 has been reported to inhibit inflammation in a rat model of inflammation (Salvemini, et.al., Science, 286, 304 (1999)), and prevent joint disease in a rat model of collagen-induced arthritis (Salvemini et al., Arthritis & Rheumatism, 44(12), 2009-2021 (2001)). Yet others of these compounds, MdPAM and MnBAM, have shown in vivo activity in the inhibition of colonic tissue injury and neutrophil accumulation into colonic tissue (Weiss et al., The Journal of Biological Chemistry, 271(42), 26149-26156 (1996)). In addition, these compounds have been reported to possess analgesic activity and to reduce inflammation and edema in the rat-paw carrageenan hyperalgesia model, see, e.g., U.S. Pat. No. 6,180,620.

Compounds of this class have also been shown to be safe and effective in the prevention and treatment of disease in human subjects. For example, GC4419 has been shown to reduce oral mucositis in head-and-neck cancer patients undergoing chemoradiation therapy (Anderson, C., Phase 1 Trial of Superoxide Dismutase (SOD) Mimetic GC4419 to Reduce Chemoradiotherapy (CRT)-Induced Mucositis (OM) in Patients (pts) with Mouth or Oropharyngeal Carcinoma (OCC), Oral Mucositis Research Workshop, MASCC/ISOO Annual Meeting on Supportive Care in Cancer, Copenhagen, Denmark (Jun. 25, 2015); Anderson, C., Phase 1b/2a Trial of Superoxide Dismutase Mimetic GC4419 to Reduce Chemoradiotherapy-Induced Oral Mucositis in Patients with Oral Cavity or Oropharyngeal Carcinoma, Int. J. of Radiation Oncol. Biol. Phys., Vol. 100, No. 2, pages 427-435 (2018)).

In addition, transition metal-containing pentaaza macrocyclic ring complexes corresponding to this class have shown efficacy in the treatment of various cancers. For example, certain compounds corresponding to this class have been provided in combination with agents such as paclitaxel and gemcitabine to enhance cancer therapies, such as in the treatment of colorectal cancer and lung cancer (non-small cell lung cancer) (see, e.g., U.S. Pat. No. 9,198,893) The 4403 compound above has also been used for treatment in in vivo models of Meth A spindle cell squamous carcinoma and RENCA renal carcinoma (Samlowski et al., Nature Medicine, 9(6), 750-755 (2003), and has also been used for treatment in in vivo models of spindle-cell squamous carcinoma metastasis (Samlowski et al., Madame Curie Bioscience Database (Internet), 230-249 (2006)). The 4419 compound above has also been used in combination with cancer therapies, such as in combination with a therapy involving administration of cisplatin and radiation, to enhance treatment in in vivo models (Sishc et al., poster for Radiation Research Society (2015)).

Accordingly, a need remains for enhanced methods of manufacture for aqueous formulations of manganese-containing coordination complexes, including formulations intended for parenteral administration of manganese-containing pentaaza macrocyclic ring complexes for the treatment of disease states. A need also remains for enhanced aqueous formulations of manganese-containing coordination complexes, to provide for parenteral administration and/or other treatment with such formulations, while maintaining acceptable stability, isotonocity, pH, and other characteristics of the formulation.

Briefly, therefore, aspects of the present disclosure are directed to a method of manufacturing an aqueous formulation of a manganese-containing coordination complex, the aqueous formulation comprising the manganese-containing coordination complex, a chloride anion, and a dianion, the method comprising combining a source of the manganese-containing coordination complex with a source of chloride anion in an aqueous solution, and simultaneously with or following combination of the source of chloride anion and the source of manganese-containing coordination complex in the aqueous solution, providing a source of a dianion to the aqueous solution to form the aqueous formulation. According to certain embodiments, the source of manganese-containing coordination complex can comprise manganese-containing component that comprises one or more of manganese in an uncoordinated state, or as coordinated to one or more ligands that are other than one or more ligands of the manganese-containing coordination complex, such as for example uncomplexed manganese remaining as an impurity from synthesis of the manganese-containing coordination complex. An amount of the source of chloride anion that is combined with the manganese-containing coordination complex is, according to certain embodiments, sufficient to provide a concentration of chloride ion in the aqueous formulation that is in excess of the concentration of dianion in the aqueous formulation.

Aspects of the disclosure are further directed to a method of treatment of a condition in a patient, comprising parenterally administering a buffered solution comprising the aqueous formulation of the manganese-containing coordination complex disclosed herein.

Aspects of the disclosure are further directed to a buffered formulation for parenteral administration of a manganese-containing pentaaza macrocyclic ring complex, the buffered formulation comprising: a buffered aqueous solution comprising: (i) the manganese-containing pentaaza macrocyclic ring complex in a concentration of from 1 mg/mL to 50 mg/mL; (ii) sodium chloride in a concentration of from 130 mM to 160 mM; and (iii) a buffering agent comprising bicarbonate in a concentration sufficient to buffer the aqueous solution to a pH in the range of 7 to 10. Storage stability of the buffered formulation is such that no manganese-containing precipitate is detectable via visual inspection for 9 months following preparation of the buffered formulation.

Other objects and features of aspects of the disclosure are described hereinafter.

### BRIEF DESCRIPTION OF THE DRAWINGS

FIG. 1 shows results for measurement of manganese via ICP-MS for day 1 following formation of aqueous formulations;

FIG. 2 shows a graphical display of the results of FIG. 1;

FIG. 3 shows results for measurement of manganese via ICP-MS for day 6 following formation of aqueous formulations;

FIG. 4 shows a graphical display of the results of FIG. 3;

FIG. 5 shows a photo of MnCO₃ crystals formed in an aqueous formulation following storage of the formulation for 9 months, as appearing in plane polarized light (lower left) and between crossed polars (upper right) (mounted in water); and

FIG. 6 shows Raman spectra collected from: (A) the MnCO₃ crystals of FIG. 5, and compared to (B) a library reference spectrum of rhodochrosite, (C) a Raman spectrum collected from a sample of MnO₂, and (D) a library reference spectrum of Hausmannite, Mn₃O₄.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

“Acyl” means a —COR moiety where R is alkyl, haloalkyl, optionally substituted aryl, or optionally substituted heteroaryl as defined herein, e.g., acetyl, trifluoroacetyl, benzoyl, and the like.

“Acyloxy” means a —OCOR moiety where R is alkyl, haloalkyl, optionally substituted aryl, or optionally substituted heteroaryl as defined herein, e.g., acetyl, trifluoroacetyl, benzoyl, and the like.

“Alkoxy” means a —OR moiety where R is alkyl as defined above, e.g., methoxy, ethoxy, propoxy, or 2-propoxy, n-, iso-, or tert-butoxy, and the like.

“Alkyl” means a linear saturated monovalent hydrocarbon moiety such as of one to six carbon atoms, or a branched saturated monovalent hydrocarbon moiety, such as of three to six carbon atoms, e.g., C₁-C₆ alkyl groups such as methyl, ethyl, propyl, 2-propyl, butyl (including all isomeric forms), pentyl (including all isomeric forms), and the like.

Moreover, unless otherwise indicated, the term “alkyl” as used herein is intended to include both “unsubstituted alkyls” and “substituted alkyls,” the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “C_{x-y}” when used in conjunction with a chemical moiety, such as alkyl and aralkyl, is meant to include groups that contain from x to y carbons in the chain. For example, the term C_{x-y} alkyl refers to substituted or unsubstituted saturated hydrocarbon groups, including straight chain alkyl and branched chain alkyl groups that contain from x to y carbon atoms in the chain.

“Alkylene” means a linear saturated divalent hydrocarbon moiety, such as of one to six carbon atoms, or a branched saturated divalent hydrocarbon moiety, such as of three to six carbon atoms, unless otherwise stated, e.g., methylene, ethylene, propylene, 1-methylpropylene, 2-methylpropylene, butylene, pentylene, and the like.

“Alkenyl” a linear unsaturated monovalent hydrocarbon moiety, such as of two to six carbon atoms, or a branched saturated monovalent hydrocarbon moiety, such as of three to six carbon atoms, e.g., ethenyl (vinyl), propenyl, 2-propenyl, butenyl (including all isomeric forms), pentenyl (including all isomeric forms), and the like.

“Alkaryl” means a monovalent moiety derived from an aryl moiety by replacing one or more hydrogen atoms with an alkyl group.

“Alkenylcycloalkenyl” means a monovalent moiety derived from an alkenyl moiety by replacing one or more hydrogen atoms with a cycloalkenyl group.

“Alkenylcycloalkyl” means a monovalent moiety derived from a cycloalkyl moiety by replacing one or more hydrogen atoms with an alkenyl group.

“Alkylcycloalkenyl” means a monovalent moiety derived from a cycloalkenyl moiety by replacing one or more hydrogen atoms with an alkyl group.

“Alkylcycloalkyl” means a monovalent moiety derived from a cycloalkyl moiety by replacing one or more hydrogen atoms with an alkyl group.

“Alkynyl” means a linear unsaturated monovalent hydrocarbon moiety, such of two to six carbon atoms, or a branched saturated monovalent hydrocarbon moiety, such as of three to six carbon atoms, e.g., ethynyl, propynyl, butynyl, isobutynyl, hexynyl, and the like.

“Alkoxy” means a monovalent moiety derived from an alkyl moiety by replacing one or more hydrogen atoms with a hydroxy group.

“Amino” means a —NR^{a}R^{b} group where R^{a} and R^{b} are independently hydrogen, alkyl or aryl.

“Aralkyl” means a monovalent moiety derived from an alkyl moiety by replacing one or more hydrogen atoms with an aryl group.

“Aryl” means a monovalent monocyclic or bicyclic aromatic hydrocarbon moiety of 6 to 10 ring atoms e.g., phenyl or naphthyl.

“Cycle” means a carbocyclic saturated monovalent hydrocarbon moiety of three to ten carbon atoms.

“Cycloalkyl” means a cyclic saturated monovalent hydrocarbon moiety of three to ten carbon atoms, e.g., cyclopropyl, cyclobutyl, cyclopentyl, or cyclohexyl, and the like.

“Cycloalkylalkyl” means a monovalent moiety derived from an alkyl moiety by replacing one or more hydrogen atoms with a cycloalkyl group, e.g., cyclopropylmethyl, cyclobutylmethyl, cyclopentylethyl, or cyclohexylethyl, and the like.

“Cycloalkylcycloalkyl” means a monovalent moiety derived from a cycloalkyl moiety by replacing one or more hydrogen atoms with a cycloalkyl group.

“Cycloalkenyl” means a cyclic monounsaturated monovalent hydrocarbon moiety of three to ten carbon atoms, e.g., cyclopropenyl, cyclobutenyl, cyclopentenyl, or cyclohexenyl, and the like.

“Cycloalkenylalkyl” means a monovalent moiety derived from an alkyl moiety by replacing one or more hydrogen atoms with a cycloalkenyl group, e.g., cyclopropenylmethyl, cyclobutenylmethyl, cyclopentenylethyl, or cyclohexenylethyl, and the like.

“Ether” means a monovalent moiety derived from an alkyl moiety by replacing one or more hydrogen atoms with an alkoxy group.

“Halo” means fluoro, chloro, bromo, or iodo, preferably fluoro or chloro.

“Heterocycle” or “heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group is a saturated ring and is not fused to aryl or heteroaryl ring as stated above, it is also referred to herein as saturated monocyclic heterocyclyl.

“Heteroaryl” means a monovalent monocyclic or bicyclic aromatic moiety of 5 to 10 ring atoms where one or more, preferably one, two, or three, ring atoms are heteroatom selected from N, O, or S, the remaining ring atoms being carbon. Representative examples include, but are not limited to, pyrrolyl, pyrazolyl, thienyl, thiazolyl, imidazolyl, furanyl, indolyl, isoindolyl, oxazolyl, isoxazolyl, benzothiazolyl, benzoxazolyl, benzimidazolyl, quinolinyl, isoquinolinyl, pyridinyl, pyrimidinyl, pyrazinyl, pyridazinyl, triazolyl, tetrazolyl, and the like.

“Nitro” means —NO₂.

“Organosulfur” means a monovalent moiety a —SR group where R is hydrogen, alkyl or aryl.

“Substituted alkyl,” “substituted cycle,” “substituted phenyl,” “substituted aryl,” “substituted heterocycle,” and “substituted nitrogen heterocycles” means an alkyl, cycle, aryl, phenyl, heterocycle or nitrogen-containing heterocycle, respectively, optionally substituted with one, two, or three substituents, such as those independently selected from alkyl, alkoxy, alkoxyalkyl, halo, hydroxy, hydroxyalkyl, or organosulfur. Generally, the term “substituted” includes groups that are substituted with any one or more of C₁₋₄alkyl, C₂₋₄alkenyl, halogen, alcohol and/or amine.

“Thioether” means a monovalent moiety derived from an alkyl moiety by replacing one or more hydrogen atoms with an —SR group wherein R is alkyl.

As used herein, (i) the compound referred to herein and in the Figures as compound 401, 4401 or GC4401 is a reference to the same compound, (ii) the compound referred to herein and in the Figures as compound 403, 4403 or GC4403 is a reference to the same compound, (iii) the compound referred to herein and in the Figures as compound 419, 4419 or GC4419 is a reference to the same compound, and (iv) the compound referred to herein and in the Figures as compound 444, 4444 or GC4444 is a reference to the same compound.

Furthermore, the use of the term “consisting essentially of,” in referring to a method of treatment, means that the method substantially does not involve providing another therapy and/or another active agent in amounts and/or under conditions that would be sufficient to provide the treatment, and which are other than the therapies and/or active agents specifically recited in the claim. Similarly, the use of the term “consisting essentially of,” in referring to a kit for treatment, means that the kit substantially does not include another therapy and/or another active agent provided in amounts and/or under conditions that would be sufficient to provide the treatment, and which are other than the therapies and/or active agents specifically recited in the claim.

### DETAILED DESCRIPTION

In one embodiment, aspects of the present disclosure are directed to a method of manufacturing an aqueous formulation of a manganese-containing coordination complex, such as an aqueous formulation for the parenteral administration of a manganese-containing pentaaza macrocyclic ring complex. Specifically, it has unexpectedly been discovered that the stability of such formulations is enhanced by carefully controlling aspects of the manufacture process. Without being limited by any one particular theory, it is believed that by providing appropriate protective anions in the formulation solution at critical points during the manufacture, the formation of manganese-containing precipitate in the formulation can be minimized, which precipitate could otherwise render the formulation unsuitable or less desirable for parenteral administration. Furthermore, while the method of manufacture is described in detail herein with respect to aqueous formulations intended for parenteral administration of manganese-containing pentaaza macrocyclic ring complexes, aspects of the invention are not limited thereto, as it is believed that similar principles also apply to aqueous formulations of other manganese-containing coordination complexes, and for uses of those aqueous formulations in areas other than for parenteral administration.

According to certain embodiments, aqueous formulations such as those used for parenteral administration can be formed by combining the manganese-containing coordination complex with a salt, such as sodium chloride, and a buffering agent, such as sodium bicarbonate or other buffering system, to provide an aqueous solution that is physiologically compatible for administration. However, it has been unexpectedly discovered that the manganese-containing coordination complex in the solution can be subject to the unwanted formation of precipitate in certain circumstances. Specifically, without being limited to any particular theory, it is believed that unwanted precipitate may form from “free” manganese or other manganese-containing impurities (as disclosed in further detail below) that are present in trace amounts as impurities of the manganese-containing coordination complex, in a case where the solution does not provide sufficient protective anions to inhibit the formation of such precipitate. Accordingly, by providing protective anions at critical points during the manufacture, the formation of unwanted precipitate can be inhibited and even prevented, to provide a sufficiently stable composition suitable for parenteral administration.

According to one aspect, it has been unexpectedly discovered that by providing chloride anions to an aqueous solution containing a manganese-containing coordination complex, such as chloride anions formed by dissolution of a chloride-containing salt in the aqueous solution, the formation of unwanted precipitate in the aqueous formulation can be inhibited. Specifically, without being limited by any theory, it is believed that the chloride anions may provide a protective effect for any “free” manganese or other manganese-containing impurity that may be present in trace amounts as an impurity of the manganese-containing coordination complex, thereby inhibiting the interaction of such “free” manganese with other anions that may be added to the solution and therefore become prone to the formation of unwanted precipitate. Again without limiting to a specific theory, it is believed that anions that may be prone to formation of unwanted precipitate with the “free” manganese or other manganese compounds may be dianions, or in other words, anions each bearing two negative charges, as opposed to the single negative charge of a chloride anion. According to certain aspects, it has been unexpectedly discovered that by providing a source of chloride anions to an aqueous solution comprising a source of manganese-containing coordination complex, either before or simultaneously with addition of a source of dianion, the formation of unwanted precipitate can be inhibited and even substantially prevented. That is, in a case where dianions are added to the aqueous formulation, it has been discovered that they are in certain embodiments ideally added either simultaneously with, or after, the protective chloride anions have been added to the aqueous formulation, such that any “free” manganese or other manganese-containing impurity may be substantially protected from interaction with the dianion, and the formation of precipitate thereby reduced and/or eliminated.

Accordingly, in certain embodiments where bicarbonate anion is provided to the aqueous solution, it has unexpectedly been found that the formation of unwanted precipitate can be inhibited and even prevented by providing a source of chloride anions before or simultaneously with addition of bicarbonate to the aqueous solution containing the manganese-containing coordination complex. Bicarbonate anion may be provided to the aqueous formulation, for example, to provide a buffering system to maintain a physiological pH of the aqueous formulation suitable for parenteral administration of the formulation. However, bicarbonate anion is in chemical equilibrium with the dianion carbonate (CO₃²⁻), which dianion, without being limited to any theory, is believed to undesirably react with “free” manganese and/or other manganese-containing impurities in the aqueous solution, and has unexpectedly been discovered to form a precipitate with such manganese over time (i.e., manganese carbonate (MnCO₃) precipitate). Surprisingly, it has been discovered that by providing the source of chloride anion either before or simultaneously with the source of dianion (e.g., bicarbonate), the formation of unwanted precipitate is inhibited, and the aqueous formulation is maintained in a stable state suitable for parenteral administration. In contrast, in a case where the source of dianion (e.g., bicarbonate) is provided before the source of chloride anion, the aqueous formulation of the manganese-containing coordination complex has been unexpectedly observed to form unwanted precipitate.

The formation of precipitate caused by addition of bicarbonate to the aqueous solution containing the manganese-containing coordination complex in the absence of chloride anions is even more surprising, as no precipitate may be immediately visually observable following formation of the aqueous formulation, and instead visible levels of precipitate formation may be only observed after a significant period of time has passed following manufacture of the aqueous formulation, such as after nine months, as is discussed in further detail in the Examples herein. Accordingly, it was unexpectedly discovered that the addition of the source of chloride anion before or simultaneously with the source of dianion (e.g., bicarbonate) was critical to inhibit or prevent precipitate formation caused by interaction of “free” manganese and/or other manganese-containing impurities with the dianion, to provide an aqueous solution that is substantially free of precipitate and thus suitable for parenteral administration.

Without being limited by any one theory, it is hypothesized that in certain embodiments, if “free” manganese or other manganese-containing impurities having a charge of +2 (Mn²⁺) (or greater) are provided in solution with a dianion, and without any chloride anions, the manganese species have sufficient binding sites available (+2) to bind the dianion (−2) (such as carbonate anion CO₃²⁻) and form the unwanted precipitate. It is further hypothesized, again without being limited to any one theory, that in a case where sufficient quantities of chloride anions having a charge of −1 are provided in solution with the “free” manganese, then the manganese-containing species and chloride ions may combine to form MnCl₂ and MnCl⁺ (and similar species for Mn³⁺ and higher oxidation states) at equilibrium in the solution, or in other words the solution at equilibrium may have quantities of manganese-containing species that are neutral or have a charge of −1, thereby reducing the number of Mn²⁺ species available for binding to the dianion, such that precipitate formation is reduced and even eliminated. Accordingly, the chloride anions may provide a protective effect when provided in sufficient amounts either before or simultaneously with addition of the dianion such as carbonate anion.

Accordingly, in one aspect of the present disclosure, a method of manufacturing is provided for the manufacture of an aqueous formulation of a manganese-containing coordination complex, where the aqueous formulation comprises the manganese-containing coordination complex, a chloride anion, and a dianion. The method generally comprises combining a source of the manganese-containing coordination complex with a source of chloride anion in an aqueous solution. The method further comprises, either simultaneously with or following combination of the source of chloride anion and the source of manganese-containing coordination complex, providing a source of a dianion to the aqueous solution to form the aqueous formulation. That is, according to one embodiment, the source of dianion is combined with the source of manganese-containing coordination complex only after or simultaneously with combination of the source of chloride anion with the source of manganese-containing coordination complex. According to certain aspects, the source of chloride anion may thus provide a protective effect to inhibit formation of precipitate cause by interaction of the dianion with “free” manganese or other manganese-containing impurities that are present in trace amounts in the source of manganese-containing coordination complex.

In one embodiment, the source of manganese-containing coordination complex comprises manganese coordinated to a macrocyclic ligand. For example, according to one embodiment, the source of manganese-containing coordination complex can comprise any one selected from the group consisting of a pentaaza macrocyclic ligand, a tetraaza macrocyclic ligand, a porphyrin macrocyclic ligand, a phthalocyanine macrocyclic ligand, and a crown ether macrocyclic ligand, among other possible macrocyclic ligands. Furthermore, according to certain embodiments, the manganese-containing coordination complex comprises manganese coordinated to one or more monodentate or polydentate ligands via nitrogen atoms of the one or more ligands. Furthermore, while in certain embodiments, the manganese-containing coordination complex comprises manganese in the +2 or +3 oxidation state (Mn(II) or Mn(III)), according to certain embodiments, the manganese-containing coordination complex comprises manganese in the +2 oxidation state (Mn(II)). In certain embodiments, as described further herein below, the manganese-containing coordination complex comprises a manganese-containing pentaaza macrocyclic ring complex, such as any described further herein, and including for example the macrocyclic ring complexes referred to herein as GC4419, GC4403, GC4711 and GC4702, among others. In one embodiment, the aqueous formulation comprises a concentration of the manganese-containing coordination complex, such as the pentaaza macrocyclic ring complex, that is at least 1 mg/mL, at least 3 mg/mL, at least 5 mg/mL, at least 9 mg/mL, at least 15 mg/mL, at least 18 mg/mL, and/or at least 20 mg/mL, but generally no more than 100 mg/mL, such as no more than 75 mg/mL, no more than 50 mg/mL, no more than 30 mg/mL, nor more than 20 mg/mL, and/or no more than 10 mg/mL. For example, the concentration of the manganese-containing coordination complex, such as the pentaaza macrocyclic ring complex, may be in a range of from 1 mg/mL to 50 mg/mL, such as in a range of from 5 mg/mL to 15 mg/mL, and even in a range of from 3 mg/mL to 10 mg/mL. For example, the aqueous formulation may comprise a pentaaza macrocyclic ring complex (e.g., GC4419) in an amount of at least 10 mg, at least 25 mg, at least 30 mg, at least 50 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, and/or at least 120 mg, but generally no more than 500 mg.

In one embodiment, the source of manganese-containing coordination complex further comprises a Mn(II)-containing component that comprises one or more of Mn(II) in an uncoordinated state (i.e., “free” Mn metal not coordinated to any ligands), or as coordinated to one or more ligands that are other than one or more ligands of the manganese-containing coordination complex. For example, the Mn(II)-containing component can comprise MnCl₂, in certain cases, or other forms of Mn(II) that are other than the manganese-containing coordination complex. Without being limited to any one theory, it is believed that this Mn(II)-containing component may contribute to the formation of precipitate in cases where the manufacturing process is not controlled as per the embodiments herein, as discussed herein. Furthermore, such Mn(II)-containing components may arise, in certain cases, during manufacture and/or synthesis of the manganese-containing coordination complex itself, and as such may be present as an otherwise relatively harmless impurity or by-product in the source of manganese-containing coordination complex. In one embodiment, the source of manganese-containing coordination complex comprises the Mn(II)-containing component, such as “free” or uncoordinated Mn(II), in a ratio by weight of the Mn(II)-containing component to the manganese-containing coordination complex that is at least 1:100,000, such as at least 1:50,000, and even at least 1:15,000, and that is no more than 1:100, such as no more than 1:1,000, no more than 1:5,000, and/or even no more than 1:8,000. For example, the ratio by weight of the Mn(II)-containing component to the manganese-containing coordination complex in the source of the manganese-containing coordination complex may be in a range of from 1:100,000 to 1:100, and/or a range of from 1:75,000 to 1:1,000, and/or a range of from 1:50,000 to 1:5,000, and/or a range of from 1:15,000 to 1:8,000. As yet another example, in a case where the aqueous formulation is prepared to provide a single dose of the manganese-containing coordination complex, such as for example for parenteral administration of a single dose of pentaaza macrocyclic ring complex, and amount of the Mn(II)-containing component (e.g., “free” Mn) may be at least 1 microgram, such as at least 10 micrograms, such as at least 50 micrograms, and even at least 100 micrograms, but typically less than about 2000 micrograms, such as less than 1000 micrograms, and even less than 850 micrograms, of the Mn(II)-containing component.

According to one embodiment, the source of chloride anion comprises a salt capable of forming chloride anions in the aqueous solution. For example, the source of chloride anion can comprise at least one selected from the group consisting of sodium chloride, potassium chloride, calcium chloride, and magnesium chloride. In one embodiment, such as for example in a case where the aqueous formulation is intended for use in parenteral administration, the source of chloride anion may be provided in a concentration and/or amount that is compatible with physiological conditions. For example, the source of chloride anion (e.g., sodium chloride) may be added in an amount sufficient to provide a concentration of chloride anion in the aqueous formulation of at least 100 mM, such as at least 110 mM, at least 115 mM, at least 120 mM, at least 130 mM, at least 145 mM and/or at least 150 mM. For example, the source of chloride anion may be provided in an amount sufficient to provide a concentration of chloride anion in the aqueous formulation of no more than 1000 mM, no more than 200 mM, no more than 180 mM, no more than 175 mM, no more than 160 mM and/or no more than 155 mM. For example, the source of chloride anion (e.g., sodium chloride) may be provided in an amount that provides a concentration of chloride anion in the aqueous formulation that is in a range of from 100 mM to 200 mM, such as a range of from 130 mM to 160 mM, and/or a range of from 145 mM to 158 mM, such as about 154 mM.

According to one embodiment, the source of dianion comprises at least one selected from the group consisting of a bicarbonate salt (e.g., sodium bicarbonate (NaHCO₃)) and a phosphate salt (e.g. sodium phosphate). In one embodiment, the source of dianion is provided in a concentration that is sufficient to provide a concentration of dianion (e.g., CO₃²⁻) of at least 0.1 mM, such as at least 0.25 mM, at least 1 mM, and/or at least 2.5 mM, and no more than 26 mM of the dianion, such as no more than 15 mM, no more than 10 mM, and/or no more than 5 mM, such as a range of dianion concentration in a range of from 0.1 mM to 15 mM, and even from 1 mM to 10 mM. As an example, in a case where bicarbonate salt is provided as the source of dianion, the concentration of bicarbonate salt provided to the aqueous formulation may be at least 5 mM, such as at least 10 mM, at least 15 mM, at least 20 mM, and/or at least 25 mM, and no more than 50 mM, such as no more than 40 mM, no more than 35 mM, and/or no more than 30 mM. For example, the concentration of a bicarbonate salt provided in the aqueous formulation may be in the range of from 5 mM to 50 mM, such as from 15 mM to 40 mM, and even from about 20 mM to 30 mM.

In yet another embodiment, a buffering system comprising one or more buffering agents may be provided to the aqueous formulation. According to certain aspects herein, the dianion itself may be a part of the buffering system, such as for example a bicarbonate buffering system and/or a phosphate buffering system, and may comprise a buffering agent that buffers a pH of the aqueous formulation in cooperation with its conjugate acid and/or base that together form the buffering system. In certain embodiments, the buffering system comprises a buffering agent that acts as a source of the dianion, and that is provided in a concentration sufficient to buffer the aqueous formulation to a predetermined pH. In one embodiment, the buffering system is provided to buffer the aqueous formulation to a physiologically acceptable pH, such as a pH within a range of from 7 to 10, and even a pH within a range of from 7.5 to 9. For example, in certain embodiments, the buffering agent may serve as a source of dianion and buffer the aqueous formulation to the predetermined pH, thereby providing a concentration of the dianion within the aqueous formulation that is consistent with buffering at that pH. In one example, in a case where the buffering agent is sodium bicarbonate, a quantity of sodium bicarbonate may be added to buffer the aqueous solution to a pH of about 8.3, with a concentration of sodium bicarbonate as added to the formulation of about 26 mM.

Furthermore, according to certain embodiments, the amount of the source of chloride anion that is provided to the aqueous formulation is such that a concentration of the chloride anion in the aqueous formulation is in excess of a concentration of the dianion in the aqueous formulation. That is, without limiting to any specific theory, a source of chloride anion may be provided in sufficient quantities such that a concentration of chloride anion exceeds that of the dianion in the aqueous formulation, which it is believed may in certain instances provide a protective effect to shield “free” Mn or other reactive Mn(II)-containing components from the dianion and thereby inhibit and/or prevent the formation of precipitate that may otherwise form via interaction with the dianion. In one embodiment, the amount of the source of chloride anion and the amount of the source of dianion are provided in the aqueous formulation in relative amounts, such that the concentration of chloride anion in the aqueous formulation exceeds the concentration of the dianion in the formulation by a ratio in mol/L of the concentration of chloride anion to dianion of at least 10:1, at least 100:1, at least 250:1, at least 500:1, at least 750:1, at least 1000:1, at least 5000:1, and/or at least 10,000:1, in the aqueous formulation.

According to one embodiment, the source of dianion may be provided to the aqueous solution for combination with the source of manganese-containing coordination complex simultaneously with the source of chloride anion. For example, in one embodiment, the source of chloride anion (e.g. sodium chloride) and the source of dianion (e.g. bicarbonate) may be combined together to form an aqueous solution. The aqueous solution may then be combined with the source of manganese-containing coordination complex, such as for example by combining the aqueous solution comprising the chloride anion and the dianion with a separate aqueous solution of the manganese-containing coordination complex, or by otherwise adding the manganese-containing coordination complex to the aqueous solution comprising the chloride anion and dianion (e.g. by dissolving the source of manganese-containing coordination complex in the aqueous solution comprising the chloride anion and dianion). For example, in one embodiment, an aqueous solution of the manganese-containing coordination complex is formed by dissolving the source of manganese-containing coordination complex in water, and optionally adjusting the pH. Another aqueous solution is prepared by combining the source of chloride anion and the source of dianion, for example in the amounts sufficient to provide predetermined concentrations of the chloride anion and dianion in the final aqueous formulation (e.g., excess chloride ion concentration). The aqueous solution of chloride anion and dianion is then added to the aqueous solution of manganese-containing coordination complex, to form the aqueous formulation comprising the manganese-containing coordination complex, the chloride anion, and the dianion, with the chloride anion being present in a concentration exceeding the concentration of the dianion in the formulation. In yet another embodiment, the aqueous solution comprising the source of chloride anion and the source of dianion is prepared, for example in the amounts sufficient to provide the predetermined concentrations of the chloride anion and dianion in the final aqueous formulation (e.g. with the concentration of chloride anions exceeding the concentration of dianion in the final aqueous formulation). The source of manganese-containing coordination complex may then be directly added and/or dissolved into the aqueous solution to provide the final aqueous formulation.

According to yet another embodiment, the source of dianion is added following combination of the source of chloride anion with the source of manganese-containing coordination complex in aqueous solution. For example, an aqueous solution of the manganese-containing coordination complex can be formed by dissolving the source of manganese-containing coordination complex in water, and optionally adjusting the pH. The source of chloride anion can be added to the aqueous solution comprising the manganese-containing coordination complex, such as by directly adding the source of chloride anion (e.g. a chloride-containing salt) to the aqueous solution to dissolve the source of chloride anion therein, and/or by providing a separate aqueous solution having the source of the chloride anion dissolved therein, and then combining the separate aqueous solution with the aqueous solution comprising the manganese-containing coordination complex. As yet another example, the source of chloride anion may be dissolved in an aqueous solution, and the source of manganese-containing coordination complex can be directly added thereto, to dissolve the manganese-containing coordination complex therein. Once the aqueous solution comprising the chloride anion and manganese-containing coordination complex has been formed, the source of dianion may be added thereto, such as for example by adding a separate aqueous solution comprising the source of dianion to the aqueous solution comprising the chloride anion and manganese-containing coordination complex, or by directly adding the source of dianion (e.g. in salt form) to the aqueous solution comprising the chloride anion and manganese-containing coordination complex. As with the simultaneous addition described above, the amounts of the source of chloride anion and the source of dianion that are provided are such that the concentration of chloride anions exceeds the concentration of dianion in the final aqueous formulation.

According to the embodiments described herein, the source of dianion is added either simultaneously or following combination of the chloride anions with the manganese-containing coordination complex. In one embodiment, substantially the entire amount of the source of dianion is added either simultaneously or following combination of the chloride anions with the manganese-containing coordination complex, such that no dianion is combined with the manganese-containing coordination in the absence of chloride anions. For example, at least 75 mol %, at least 85 mol %, at least 90 mol %, at least 95 mol %, at least 98 mol %, at least 99 mol %, and/or the entire molar amount of the source of dianion that is added to form the aqueous formulation is added simultaneously with or following combination of the source of chloride anion with the manganese-containing coordination complex. According to one embodiment, no amount of the source of dianion is combined with the manganese-containing coordination complex, unless an excess of chloride anions is already present in, or is being simultaneously added to, the aqueous solution containing the manganese-containing coordination complex. Furthermore, according to certain embodiments where the source of dianion is provided following the combination of the chloride anions and manganese-containing coordination complex in an aqueous solution, the source of dianion (e.g. bicarbonate salt) may be added thereto at least 30 seconds, at least 1 minute, at least 5 minutes, at least 10 minutes, at least 30 minutes, and/or at least one hour after combination of the manganese-containing coordination complex with the source of chloride anion in the aqueous solution.

In one embodiment of a method of manufacture according to aspects described herein, the pH of filtered, de-ionized water suitable for injection purposes is brought to a pH of about 7.5 using NaOH. A manganese-containing coordination complex, such as the pentaaza macrocyclic ring complex corresponding to GC4419, or other pentaaza macrocyclic ring complex described herein, is added in an amount of 9 mg/mL to the water to form an aqueous solution thereof. Sodium chloride salt is added to the aqueous solution to provide a 0.9% by weight solution. Following addition of the sodium chloride salt, sodium bicarbonate is added to the aqueous solution to buffer the solution, in an amount of 26 mM of the sodium bicarbonate salt. The resulting aqueous formulation comprises good stability and shelf life, with no visibly observable formation of precipitate after 9 months under appropriate storage conditions, including maintaining at a temperature of about 5-8° C., as discussed further in the Examples herein.

According to certain embodiments, the aqueous formulation may be used in a method of treatment of a condition in a patient. For example, the aqueous formulation may be used for parenterally administering a buffered solution comprising the aqueous formulation of the manganese-containing coordination complex. In one example, the aqueous formulation may be used for intravenously administering the manganese-containing coordination complex. Further methods of treatment using the aqueous formulation, and disease states and conditions that may be treatable therewith, are discussed in further detail herein below.

According to yet a further embodiment, aspects of the disclosure relate to a buffered formulation comprising the aqueous formulation, such as a buffered formulation of a manganese-containing pentaaza macrocyclic ring complex. The buffered formulation can comprise, for example, an aqueous formulation prepared according to any of the manufacturing embodiments described herein. According to one embodiment, the buffered aqueous solution can comprise (i) a pentaaza macrocyclic ring complex in a concentration of from 2 mM to 100 mM, (ii) sodium chloride in a concentration of from 130 mM to 160 mM, and a buffering agent comprising sodium bicarbonate in a concentration sufficient to buffer the aqueous solution to a pH in the range of 7 to 10, such as for example in a concentration of from 20 mM to 30 mM. For example, according to one embodiment, the buffered aqueous solution can comprise the pentaaza macrocyclic ring complex in a concentration of at least 2 mM, at least 6 mM, at least 18 mM, at least 20 mM, and/or at least 40 mM, but less than 100 mM.

For example, according to one embodiment, the buffered aqueous solution can comprise the pentaaza macrocyclic ring complex in a concentration of at least 1 mg/mL, at least 3 mg/mL, at least 5 mg/mL, at least 9 mg/mL, at least 15 mg/mL, at least 18 mg/mL, and/or at least 20 mg/mL, but generally no more than 100 mg/mL, such as no more than 75 mg/mL, no more than 50 mg/mL, no more than 30 mg/mL, nor more than 20 mg/mL, and/or no more than 10 mg/mL. For example, the concentration of the pentaaza macrocyclic ring complex may be in a range of from 1 mg/mL to 50 mg/mL, such as in a range of from 5 mg/mL to 15 mg/mL, and even in a range of from 3 mg/mL to 10 mg/mL. For example, the aqueous formulation may comprise a pentaaza macrocyclic ring complex (e.g., GC4419) in an amount of at least 10 mg, at least 25 mg, at least 30 mg, at least 50 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, and/or at least 120 mg, but generally no more than 500 mg.

When manufactured as described herein (e.g. by combining the sodium bicarbonate either simultaneously or following combination of the sodium chloride with the pentaaza macrocyclic ring complex), the buffered formulation can exhibit good storage stability. For example, in one embodiment, the storage stability of the buffered formulation is such that no manganese-containing precipitate is discernible via visual detection for 9 months following preparation of the buffered formulation. In yet another embodiment, the storage stability is of the buffered formulation such that no manganese-containing precipitate is discernible via visual detection after 1 day and/or after 6 days following formation of the buffered formulation. The visual detection can comprise visual inspection of the buffered solution to identify whether any precipitate has formed in the solution. According to further embodiments, the buffered formulation may be prepared according to any of the methods described herein.

As yet another example, an ICP-MS (inductively coupled plasma-mass spectrometry) storage stability assay can be performed to determine an amount of manganese-containing precipitate that is generated over time in the buffered formulation. According to one embodiment, a ICP-MS storage stability assay can comprise filtering the buffered formulation through a 0.45 micrometer filter, washing the filter with pH 8.0 water, digesting the filter contents with nitric acid, and performing inductively coupled mass-spectrometry (ICP-MS) to detect manganese content of any precipitate. According to embodiments herein, the amount of manganese measured by the ICP-MS storage stability assay after at least 1 day, at least 6 days, and/or at least 9 months is less than 1500 ppm, and/or even less than 1200 ppm.

Further details and/or embodiments of the aqueous formulation are provided below, including further description of components of the aqueous formulation, and optional additives thereto, as well as methods of treatment therewith.

### Manganese-Containing Pentaaza Macrocyclic Ring Complex

In one embodiment, the pentaaza macrocyclic ring complex corresponds to the complex of Formula (I):

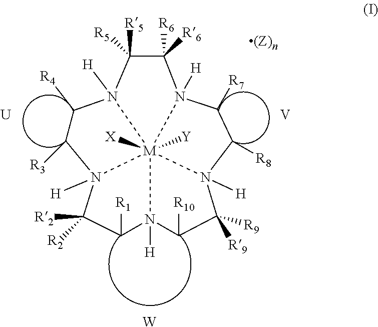

wherein
M is Mn²⁺ or Mn³⁺,
R₁, R₂, R′₂, R₃, R₄, R₅, R′₅, R₆, R′₆, R₇, R₈, R₉, R′₉, and R₁₀ are independently hydrogen, hydrocarbyl, substituted hydrocarbyl, heterocyclyl, an amino acid side chain moiety, or a moiety selected from the group consisting
of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂),
and —OP(O)(OR₁₁)(OR₁₂), wherein R₁₁ and R₁₂ are independently hydrogen or alkyl;
U, together with the adjacent carbon atoms of the macrocycle, forms a fused substituted or unsubstituted, saturated, partially saturated or unsaturated, cycle or heterocycle having 3 to 20 ring carbon atoms;
V, together with the adjacent carbon atoms of the macrocycle, forms a fused substituted or unsubstituted, saturated, partially saturated or unsaturated, cycle or heterocycle having 3 to 20 ring carbon atoms;
W, together with the nitrogen of the macrocycle and the carbon atoms of the macrocycle to which it is attached, forms an aromatic or alicyclic, substituted or unsubstituted, saturated, partially saturated or unsaturated nitrogen-containing fused heterocycle having 2 to 20 ring carbon atoms, provided that when W is a fused aromatic heterocycle the hydrogen attached to the nitrogen which is both part of the heterocycle and the macrocycle and R₁ and R₁₀ attached to the carbon atoms which are both part of the heterocycle and the macrocycle are absent;
X and Y represent suitable ligands which are derived from any monodentate or polydentate coordinating ligand or ligand system or the corresponding anion thereof;
Z is a counterion;
n is an integer from 0 to 3; and
the dashed lines represent coordinating bonds between the nitrogen atoms of the macrocycle and the transition metal, manganese.

As noted above in connection with the pentaaza macrocyclic ring complex of Formula (I), M is Mn²⁺ or Mn³⁺. In one particular embodiment in which the pentaaza macrocyclic ring complex corresponds to Formula (I), M is Mn²⁺. In another particular embodiment in which the pentaaza macrocyclic ring complex corresponds to Formula (I), M is Mn³⁺.

In the embodiments in which one or more of R₁, R₂, R′₂, R₃, R₄, R₅, R′₅, R₆, R′₆, R₇, R₈, R₉, R′₉, and R₁₀ are hydrocarbyl, for example, suitable hydrocarbyl moieties include, but are not limited to alkenyl, alkenylcycloalkenyl, alkenylcycloalkyl, alkyl, alkylcycloalkenyl, alkylcycloalkyl, alkynyl, aralkyl, aryl, cycloalkenyl, cycloalkyl, cycloalkylalkyl, cycloalkylcycloalkyl, cycloalkenylalkyl, and aralkyl. In one embodiment, R₁, R₂, R′₂, R₃, R₄, R₅, R′₅, R₆, R′₆, R₇, R₈, R₉, R′₉, and R₁₀ are independently hydrogen, hydrocarbyl, substituted hydrocarbyl, or heterocyclyl. More preferably in this embodiment, R₁, R₂, R′₂, R₃, R₄, R₅, R′₅, R₆, R′₆, R₇, R₈, R₉, R′₉, and R₁₀ are independently hydrogen or lower alkyl (e.g., C₁-C₆ alkyl, more typically C₁-C₄ alkyl). Thus, for example, R₁, R₂, R′₂, R₃, R₄, R₅, R′₅, R₆, R′₆, R₇, R₈, R₉, R′₉, and R₁₀ may be independently hydrogen, methyl, ethyl, propyl, or butyl (straight, branched, or cyclic). In one preferred embodiment, R₁, R₂, R′₂, R₃, R₄, R₅, R′₅, R₆, R′₆, R₇, R₈, R₉, R′₉, and R₁₀ are independently hydrogen or methyl.

In one preferred embodiment in which the pentaaza macrocyclic ring complex corresponds to Formula (I), R₁, R₂, R′₂, R₃, R₄, R₅, R′₅, R₇, R₈, R₉, R′₉, and R₁₀ are each hydrogen and one of R₆ and R′₆ is hydrogen and the other of R₆ and R′₆ is methyl. In this embodiment, for example, R₁, R₂, R′₂, R₃, R₄, R₅, R′₅, R₆, R₇, R₈, R₉, R′₉, and R₁₀ may each be hydrogen while R′₆ is methyl. Alternatively, for example, R₁, R₂, R′₂, R₃, R₄, R₅, R′₅, R′₆, R₇, R₈, R₉, R′₉, and R₁₀ may each be hydrogen while R₆ is methyl. In another preferred embodiment in which the pentaaza macrocyclic ring complex corresponds to Formula (I), R₁, R₃, R₄, R₅, R′₅, R′₆, R₇, R₈, and R₁₀ are each hydrogen, one of R₂ and R′₂ is hydrogen and the other of R₂ and R′₂ is methyl, and one of R₉ and R′₉ is hydrogen and the other of R₉ and R′₉ is methyl. In this embodiment, for example, R₁, R′₂, R₃, R₄, R₅, R′₅, R₇, R₈, R₉, and R₁₀ may each be hydrogen while R₂ and R¹9 are methyl. Alternatively, for example, R₁, R₂, R₃, R₄, R₅, R′₅, R₇, R₈, R′₉, and R₁₀ may each be hydrogen while R′₂ and R₉ are methyl. In another embodiment in which the pentaaza macrocyclic ring complex corresponds to Formula (I), R₁, R₂, R′₂, R₃, R₄, R₅, R′₅, R₆, R′₆, R₇, R₈, R₉, R′₉, and R₁₀ are each hydrogen.

In certain embodiments the U and V moieties are independently substituted or unsubstituted fused cycloalkyl moieties having 3 to 20 ring carbon atoms, more preferably 4 to 10 ring carbon atoms. In a particular embodiment, the U and V moieties are each trans-cyclohexanyl fused rings.

In certain embodiments the W moiety is a substituted or unsubstituted fused heteroaromatic moiety. In a particular embodiment, the W moiety is a substituted or unsubstituted fused pyridino moiety. Where W is a substituted fused pyridino moiety, for example, the W moiety is typically substituted with a hydrocarbyl or substituted hydrocarbyl moiety (e.g., alkyl, substituted alkyl) at the ring carbon atom positioned para to the nitrogen atom of the heterocycle. In a one preferred embodiment, the W moiety is an unsubstituted fused pyridino moiety.

As noted above, X and Y represent suitable ligands which are derived from any monodentate or polydentate coordinating ligand or ligand system or the corresponding anion thereof (for example benzoic acid or benzoate anion, phenol or phenoxide anion, alcohol or alkoxide anion). For example, X and Y may be selected from the group consisting of halo, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof, among other possibilities. In one embodiment, X and Y if present, are independently selected from the group consisting of halo, nitrate, and bicarbonate ligands. For example, in this embodiment, X and Y, if present, are halo ligands, such as chloro ligands.

Furthermore, in one embodiment X and Y correspond to —O—C(O)—X₁, where each X₁ is —C(X₂)(X₃)(X₄), and each X₁ is independently substituted or unsubstituted phenyl or —C(—X₂)(—X₃)(—X₄);
each X₂ is independently substituted or unsubstituted phenyl, methyl, ethyl or propyl;
each X₃ is independently hydrogen, hydroxyl, methyl, ethyl, propyl, amino, —X₅C(═O)R₁₃ where X₅ is NH or O, and R₁₃ is C₁-C₁₈ alkyl, substituted or unsubstituted aryl or C₁-C₁₈ aralkyl, or —OR₁₄, where R₁₄ is C₁-C₁₈ alkyl, substituted or unsubstituted aryl or C₁-C₁₈ aralkyl, or together with X₄ is (═O); and
each X₄ is independently hydrogen or together with X₃ is (═O).

In yet another embodiment, X and Y are independently selected from the group consisting of charge-neutralizing anions which are derived from any monodentate or polydentate coordinating ligand and a ligand system and the corresponding anion thereof; or X and Y are independently attached to one or more of R₁, R₂, R′₂, R₃, R₄, R₅, R′₅, R₆, R′₆, R₇, R₈, R₉, R′₉, and R₁₀.

In the pentaaza macrocyclic ring complex corresponding to Formula (I), Z is a counterion (e.g., a charge-neutralizing anion), wherein n is an integer from 0 to 3. In general, Z may correspond to counterions of the moieties recited above in connection for X and Y.

In combination, among certain preferred embodiments are pentaaza macrocyclic ring complexes corresponding to Formula (I) wherein
M is Mn²⁺ or Mn³⁺,
R₁, R₂, R′₂, R₃, R₄, R₅, R′₅, R₆, R′₆, R₇, R₈, R₉, R′₉, and R₁₀ are independently hydrogen or lower alkyl;
U and V are each trans-cyclohexanyl fused rings;
W is a substituted or unsubstituted fused pyridino moiety;
X and Y are ligands; and
Z, if present, is a charge-neutralizing anion.

More preferably in these embodiments, M is Mn²⁺, R₁, R₂, R′₂, R₃, R₄, R₅, R′₅, R₆, R′₆, R₇, R₈, R₉, R′₉, and R₁₀ are independently hydrogen or methyl; U and V are each trans-cyclohexanyl fused rings; W is an unsubstituted fused pyridino moiety; and X and Y are independently halo ligands (e.g., fluoro, chloro, bromo, iodo). Z, if present, may be a halide anion (e.g., fluoride, chloride, bromide, iodide).

In yet another embodiment, the pentaaza macrocyclic ring complex is represented by Formula (II) below:

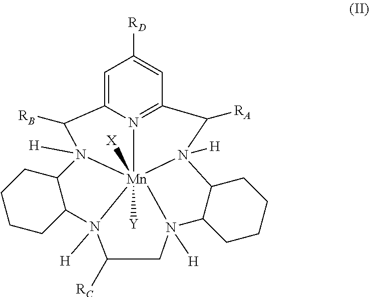

wherein
X and Y represent suitable ligands which are derived from any monodentate or polydentate coordinating ligand or ligand system or the corresponding anion thereof; and
R_{A}, R_{B}, R_{C}, and R_{D} are independently hydrogen, hydrocarbyl, substituted hydrocarbyl, heterocyclyl, an amino acid side chain moiety, or a moiety selected from the group consisting
of —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂),
and —OP(O)(OR₁₁)(OR₁₂), wherein R₁₁ and R₁₂ are independently hydrogen or alkyl.

Furthermore, in one embodiment, the pentaaza macrocyclic ring complex is represented by Formula (Ill) or Formula (IV):

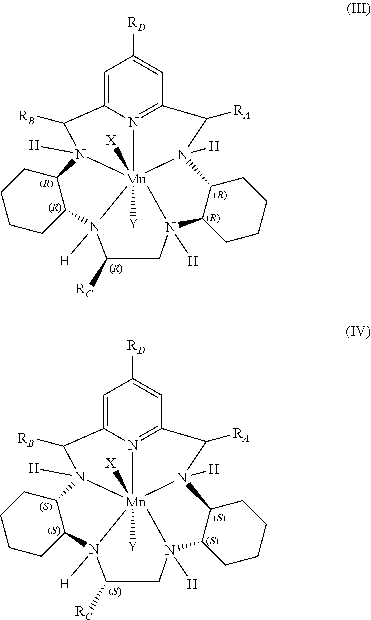

wherein
X and Y represent suitable ligands which are derived from any monodentate or polydentate coordinating ligand or ligand system or the corresponding anion thereof; and
R_{A}, R_{B}, R_{C}, and R_{D} are independently hydrogen, hydrocarbyl, substituted hydrocarbyl, heterocyclyl, an amino acid side chain moiety, or a moiety selected from the group consisting
of —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂),
and —OP(O)(OR₁₁)(OR₁₂), wherein R₁₁ and R₁₂ are independently hydrogen or alkyl.

In yet another embodiment, the pentaaza macrocyclic ring complex is a compound represented by a formula selected from the group consisting of Formulae (V)-(XVI):

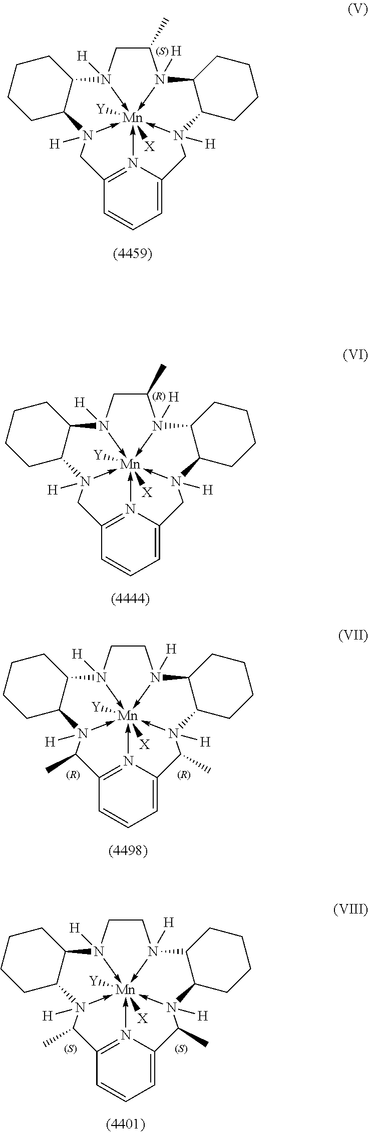

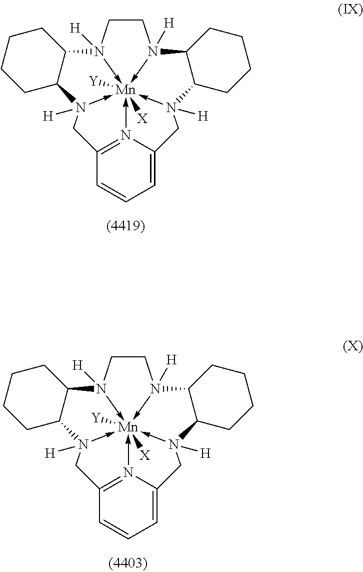

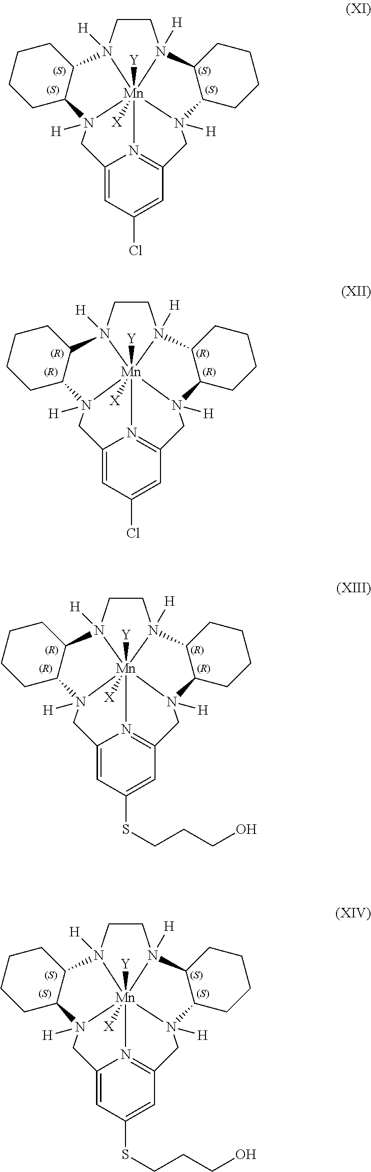

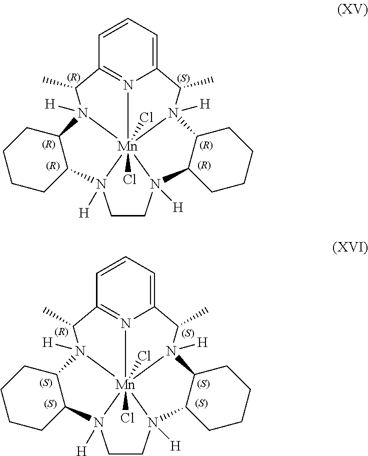

In one embodiment, X and Y in any of the formulae herein are independently selected from the group consisting of fluoro, chloro, bromo and iodo anions. In yet another embodiment, X and Y in any of the formulae herein are independently selected from the group consisting of alkyl carboxylates, aryl carboxylates and arylalkyl carboxylates. In yet another embodiment, X and Y in any of the formulae herein are independently amino acids.

In one embodiment, the pentaaza macrocyclic ring complex has the following Formula (IA):

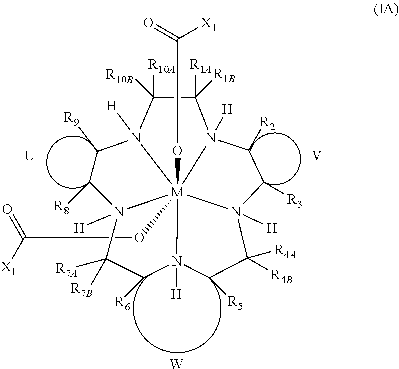

wherein
M is Mn²⁺ or Mn³⁺,
R_{1A}, R_{1B}, R₂, R₃, R_{4A}, R_{4B}, R₅, R₆, R_{7A}, R_{7B}, R₈, R₉, R_{10A}, and R_{10B} are independently hydrogen, hydrocarbyl, substituted hydrocarbyl, heterocyclyl, an amino acid side chain moiety, or a moiety independently selected from the group consisting of —OR₁₁, —NR₁₁R₁₂, —COR₁₁, —CO₂R₁₁, —C(═O) NR₁₁R₁₂, —SR₁₁, —SOR₁₁, —SO₂R₁₁, —SO₂NR₁₁R₁₂, —N(OR₁₁)(R₁₂), —P(═O)(OR₁₁)(OR₁₂), —P(═O)(OR₁₁)(R₁₂), and —OP(═O)(OR₁₁)(OR₁₂), wherein R₁₁ and R₁₂ are independently hydrogen or alkyl;
U, together with the adjacent carbon atoms of the macrocycle, forms a fused substituted or unsubstituted, saturated, partially saturated or unsaturated, cycle or heterocycle having 3 to 20 ring carbon atoms;
V, together with the adjacent carbon atoms of the macrocycle, forms a fused substituted or unsubstituted, saturated, partially saturated or unsaturated, cycle or heterocycle having 3 to 20 ring carbon atoms;
W, together with the nitrogen of the macrocycle and the carbon atoms of the macrocycle to which it is attached, forms an aromatic or alicyclic, substituted or unsubstituted, saturated, partially saturated or unsaturated nitrogen-containing fused heterocycle having 2 to 20 ring carbon atoms, provided that when W is a fused aromatic heterocycle the hydrogen attached to the nitrogen which is both part of the heterocycle and the macrocycle and R₅ and R₆ attached to the carbon atoms which are both part of the heterocycle and the macrocycle are absent; wherein
each X₁ is independently substituted or unsubstituted phenyl or —C(—X₂)(—X₃)(—X₄);
each X₂ is independently substituted or unsubstituted phenyl or alkyl;
each X₃ is independently hydrogen, hydroxyl, alkyl, amino, —X₅C(═O)R₁₃ where X₅ is NH or O, and R₁₃ is C₁-C₁₈ alkyl, substituted or unsubstituted aryl or C₁-C₁₈ aralkyl, or —OR₁₄, where R₁₄ is C₁-C₁₈alkyl, substituted or unsubstituted aryl or C₁-C₁₈ aralkyl, or together with X₄ is (═O);
each X₄ is independently hydrogen or together with X₃ is (═O); and
the bonds between the transition metal M and the macrocyclic nitrogen atoms and the bonds between the transition metal M and the oxygen atoms of the axial ligands —OC(═O)X₁ are coordinate covalent bonds.

In one embodiment, within Formula (IA), and groups contained therein, in one group of compounds X₁ is —C(—X₂)(—X₃)(—X₄) and each X₂, X₃, and X₄, in combination, corresponds to any of the combinations identified in the following table:

| Combination | X₂ | X₃ | X₄ |
|---|---|---|---|
| 1 | Ph | H | H |
| 2 | Ph | OH | H |
| 3 | Ph | NH₂ | H |
| Combination | X₂ | X₃ | X₄ |
| 4 | Ph | ═O | |
| | | (X₃ and X₄ in | |
| | | combination) | |
| 5 | Ph | CH₃ | H |
| 6 | CH₃ | H | H |
| 7 | CH₃ | OH | H |
| 8 | CH₃ | NH₂ | H |

| | | |
|---|---|---|
| 9 | CH₃ | ═O |
| | | (X₃ and X₄ in |
| | | combination) |

Furthermore, within embodiment (IA), and groups contained therein, in one group of compounds X₁ is C(—X₂)(—X₃)(—X₄), and X₃ is —X₅C(═O)R₁₃, such that the combinations of X₂, X₃ and X₄ include any of the combinations identified in the following table:

| Combination | X₂ | X₃ | X₄ |
|---|---|---|---|
| 1 | Ph | NHC(═O)R₁₃ | H |
| 2 | Ph | OC(═O)R₁₃ | H |
| 3 | CH₃ | NHC(═O)R₁₃ | H |
| 4 | CH₃ | OC(═O)R₁₃ | H |

where R₁₃ is C₁-C₁₈ alkyl, substituted or unsubstituted aryl or C₁-C₁₈ aralkyl, or —OR₁₄, where R₁₄ is C₁-C₁₈ alkyl, substituted or unsubstituted aryl or C₁-C₁₈ aralkyl.

In one embodiment, the pentaaza macrocyclic ring complex corresponding to Formula (IA) is one of the complexes Formula (IE), such as (IE_{R1}), (IE_{S1}), (IE_{R2}), (IE_{S2}), (IE_{R3}), or (IE_{S3}):

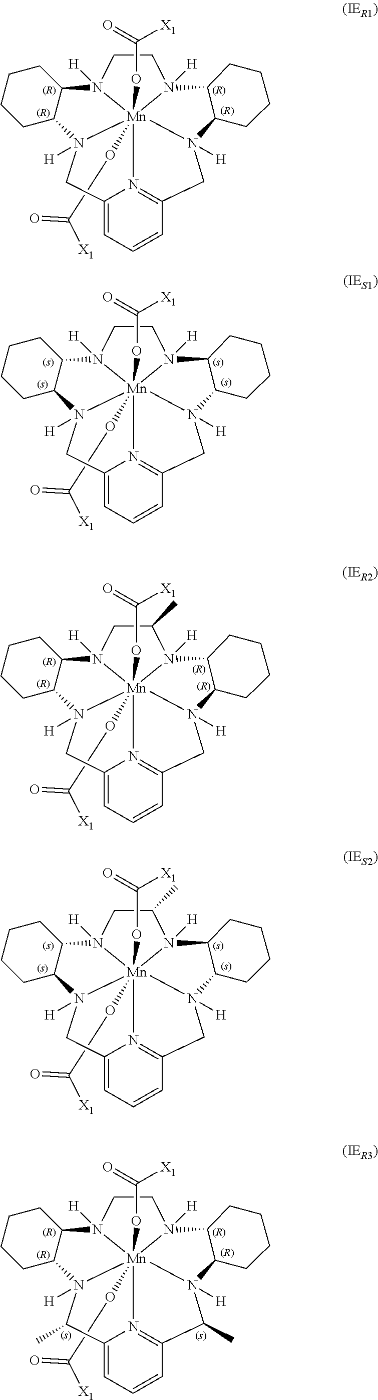

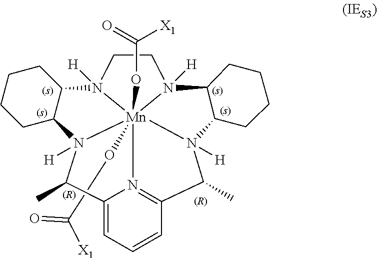

wherein
M is Mn⁺² or Mn⁺³,
each X₁ is independently substituted or unsubstituted phenyl or —C(X₂)(X₃)(X₄);
each X₂ is independently substituted or unsubstituted phenyl, methyl, ethyl, or propyl;
each X₃ is independently hydrogen, hydroxyl, methyl, ethyl, propyl, amino, or together with X₄ is ═O;
each X₄ is independently hydrogen or together with X₃ is ═O; and
the bonds between the manganese and the macrocyclic nitrogen atoms and the bonds between the manganese and the oxygen atoms of the axial ligands —OC(O)X₁ are coordinate covalent bonds.

In one embodiment, each X₁ is —C(X₂)(X₃)(X₄) and each —C(X₂)(X₃)(X₄) corresponds to any of combinations 1 to 9 appearing in the table for Formula (IA) above.

In yet another embodiment, the X and Yin pentaaza macrocyclic ring complex of Formula (I) correspond to the ligands in Formulas (IA) or (IE). For example, X and Y in the complex of Formula (I) may correspond to —O—C(O)—X₁, where X₁ is as defined for the complex of Formula (IA) and (IE) above.

In one embodiment, the pentaaza macrocyclic ring complexes corresponding to Formula (I) (e.g., of Formula (I) or any of the subsets of Formula (I) corresponding to Formula (II)-(XIV), (IA) and (IE)), can comprise any of the following structures:

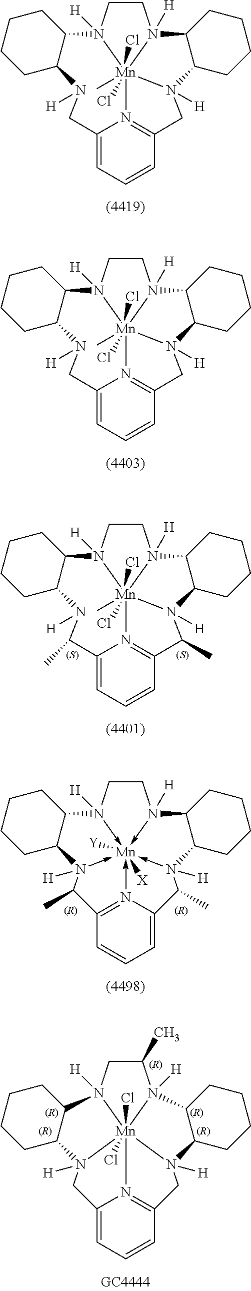

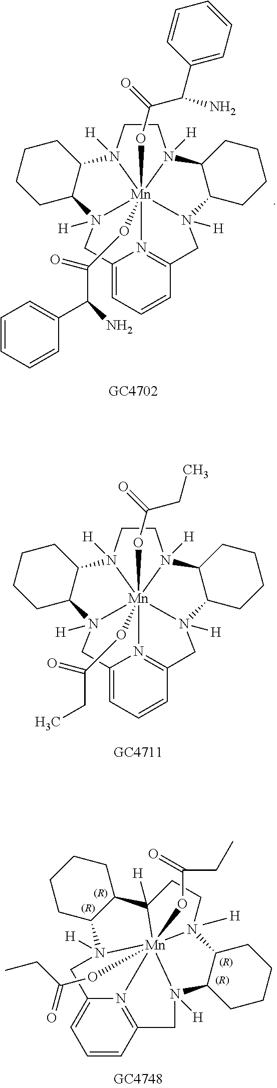

In one embodiment, the pentaaza macrocyclic ring complexes for use in the methods and compositions described herein include those corresponding to Formulae (2), (3), (4), (5), (6), and (7):

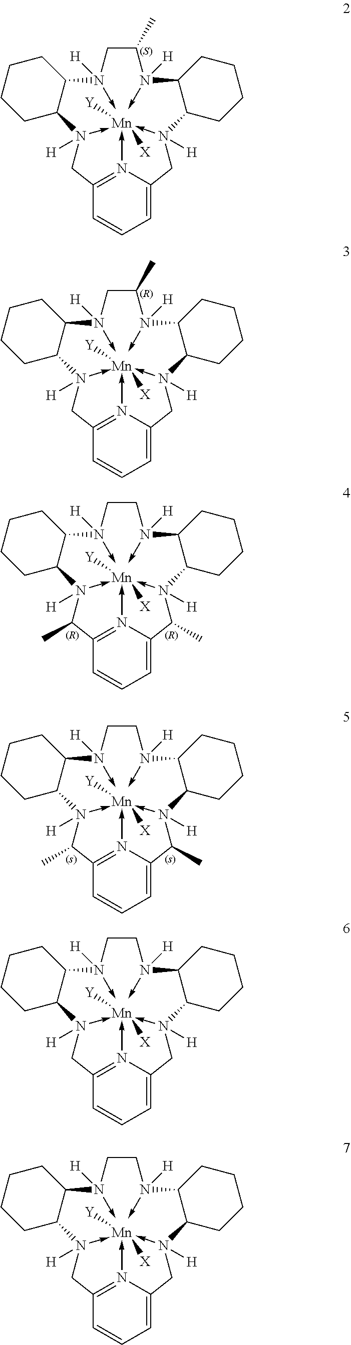

wherein X and Y in each of Formulae (2), (3), (4), (5), (6), and (7) are independently ligands. For example, according to one embodiment, the pentaaza macrocyclic ring complex for use in the methods and compositions described herein include those corresponding to Formulae (2), (3), (4), (5), (6), and (7) with X and Y in each of these formulae being halo, such as chloro. Alternatively, X and Y may be ligands other than chloro, such as any of the ligands described above.

In another embodiment, the pentaaza macrocyclic ring complex corresponds to Formula (6) or Formula (7):

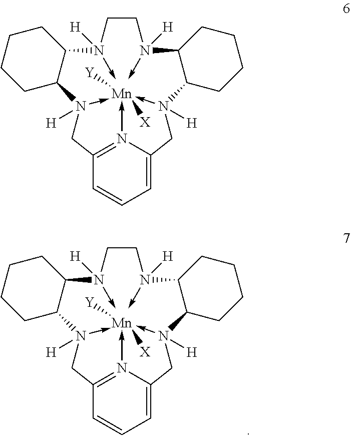

The chemical structures of 6 (such as the dichloro complex form described, for example, in Riley, D. P., Schall, O. F., 2007, Advances in Inorganic Chemistry, 59: 233-263) and of 7 herein (such as the dichloro complex form of 7), are identical except that they possess mirror image chirality; that is, the enantiomeric structures are non-superimposable.

For example, the pentaaza macrocyclic ring complex may correspond to at least one of the complexes below:

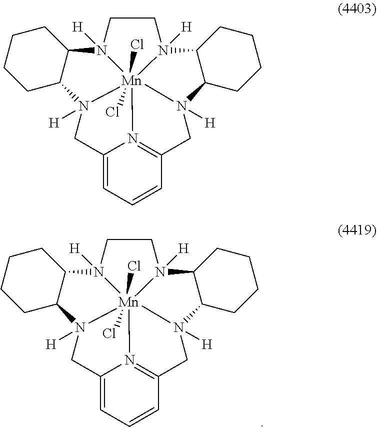

In yet another embodiment, the pentaaza macrocyclic ring complex may correspond to at least one of the complexes below, and/or an enantiomer thereof:

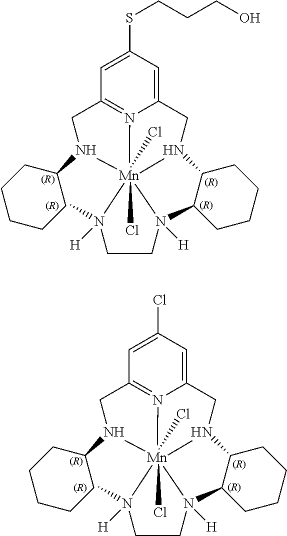

In one embodiment, the enantiomeric purity of the pentaaza macrocyclic ring complex is greater than 95%, more preferably greater than 98%, more preferably greater than 99%, and most preferably greater than 99.5%. As used herein, the term “enantiomeric purity” refers to the amount of a compound having the depicted absolute stereochemistry, expressed as a percentage of the total amount of the depicted compound and its enantiomer. In one embodiment, the diastereomeric purity of the pentaaza macrocyclic ring complex is greater than 98%, more preferably greater than 99%, and most preferably greater than 99.5%. As used herein, the term “diastereomeric purity” refers to the amount of a compound having the depicted absolute stereochemistry, expressed as a percentage of the total amount of the depicted compound and its diastereomers. Methods for determining diastereomeric and enantiomeric purity are well-known in the art. Diastereomeric purity can be determined by any analytical method capable of quantitatively distinguishing between a compound and its diastereomers, such as high-performance liquid chromatography (HPLC). Similarly, enantiomeric purity can be determined by any analytical method capable of quantitatively distinguishing between a compound and its enantiomer. Examples of suitable analytical methods for determining enantiomeric purity include, without limitation, optical rotation of plane-polarized light using a polarimeter, and HPLC using a chiral column packing material.

In one embodiment, a therapeutically effective amount of the pentaaza macrocyclic ring complex may be an amount sufficient to provide a peak plasma concentration of at least 0.1 μM when administered to a patient. For example, in one embodiment, the pentaaza macrocyclic ring complex may be administered in an amount sufficient to provide a peak plasma concentration of at least 1 μM when administered to a patient. In yet another embodiment, the pentaaza macrocyclic ring complex may be administered in an amount sufficient to provide a peak plasma concentration of at least 10 μM when administered to a patient. Generally, the pentaaza macrocyclic ring complex will not be administered in an amount that would provide a peak plasma concentration greater than 40 μM when administered to a patient. For example, the pentaaza macrocyclic ring complex may be administered in an amount sufficient to provide a peak plasma concentration in the range of from 0.1 μM to 40 μM in a patient. As another example, the pentaaza macrocyclic ring complex may be administered in an amount sufficient to provide a peak plasma concentration in the range of from 0.5 μM to 20 μM in a patient. As another example, the pentaaza macrocyclic ring complex may be administered in an amount sufficient to provide a peak plasma concentration in the range of from 1 μM to 10 μM in a patient.

In yet another embodiment, a dose of the pentaaza macrocyclic ring complex that is administered per kg body weight of the patient may be at least 0.1 mg/kg, such as at least 0.2 mg/kg. For example, the dose of the pentaaza macrocyclic ring complex that is administered per kg body weight of the patient may be at least 0.5 mg/kg. As another example, the dose of the pentaaza macrocyclic ring complex that is administered per kg body weight of the patient may be at least 1 mg/kg. In another example, the pentaaza macrocyclic compound that is administered per kg body weight may be at least 2 mg/kg, such as at least 3 mg/kg, and even at least about 15 mg/kg, such as at least 24 mg/kg and even at least 40 mg/kg. Generally, the dose of the pentaaza macrocyclic ring complex that is administered per kg body weight of the patient will not exceed 1000 mg/kg. For example the dose of the pentaaza macrocyclic ring complex that is administered per kg body weight of the patient may be in the range of from 0.1 to 1000 mg/kg, such as from 0.2 mg/kg to 40 mg/kg, such as 0.2 mg/kg to 24 mg/kg, and even 0.2 mg/kg to 10 mg/kg. As another example, the dose of the pentaaza macrocyclic ring complex that is administered per kg body weight may be in a range of from 1 mg/kg to 1000 mg/kg, such as from 3 mg/kg to 1000 mg/kg, and even from 5 mg/kg to 1000 mg/kg, such as 10 mg/kg to 1000 mg/kg. As another example, the dose of the pentaaza macrocyclic ring complex that is administered per kg body weight may be in a range of from 2 mg/kg to 15 mg/kg. As yet another example, the dose of the pentaaza macrocyclic ring complex that is administered per kg body weight may be in a range of from 3 mg/kg to 10 mg/kg. As another example, the dose of the pentaaza macrocyclic ring complex that is administered per kg body weight of the patient may be in the range of from 0.5 to 5 mg/kg. As yet a further example, the dose of the pentaaza macrocyclic ring complex that is administered per kg body weight of the patient may be in the range of from 1 to 5 mg/kg.

In one embodiment, the dose of the pentaaza macrocyclic ring complex may be at least 15 mg, at least 30 mg, at least 50 mg, at least 75 mg, at least 90 mg, at least 100 mg and/or at least 112 mg. The dose of pentaaza macrocyclic ring complex may also be administered over a predetermined period of infusion, such as a dosing rate for an infusion period of 15 minutes, 30 minutes, 45 minutes, 60 minutes, and/or a longer infusion duration. According to one embodiment, the pentaaza macrocyclic ring complex such as GC4419 may be administered at an infusion rate equivalent to at least 75 mg and/or at least 90 mg over course of an hour.

In one embodiment, the dosages and/or plasma concentrations discussed above may be particularly suitable for the pentaaza macrocyclic ring complex corresponding to GC4419, although they may also be suitable for other pentaaza macrocyclic ring complexes. In addition, one or ordinary skill in the art would recognize how to adjust the dosages and/or plasma concentrations based on factors such as the molecular weight and/or activity of the particular compound being used. For example, for a pentaaza macrocyclic ring complex having an activity twice that of GC4419, the dosage and/or plasma concentration may be halved, or for a pentaaza macrocyclic ring complex having a higher molecular weight that GC4419, a correspondingly higher dosage may be used.

The dosing schedule of the pentaaza macrocyclic ring complex can similarly be selected according to the intended treatment. For example, in one embodiment, a suitable dosing schedule can comprise dosing a patient at least once per week, such as at least 2, 3, 4, 5, 6 or 7 days per week (e.g., daily), during a course of treatment. As another example, in one embodiment, the dosing may be at least once a day (qd), or even at least twice a day (bid).

### Methods of Treatment

Treatment of conditions including oral mucositis, cancer, or other conditions described herein includes achieving a therapeutic benefit, however the therapy may also be administered to achieve a prophylactic benefit. Therapeutic benefits generally refer to at least a partial eradication or amelioration of the underlying disorder being treated. For example, in a cancer patient, therapeutic benefit includes (partial or complete) eradication or amelioration of the underlying cancer. Also, a therapeutic benefit is achieved with at least partial, or complete, eradication or amelioration of one or more of the physiological symptoms associated with the underlying disorder such that an improvement is observed in the patient, notwithstanding the fact that the patient may still be afflicted with the underlying disorder. For prophylactic benefit, a method of the disclosure may be performed on, or a composition of the invention administered to, a patient at risk of developing cancer, or to a patient reporting one or more of the physiological symptoms of such conditions, even though a diagnosis of the condition may not have been made.

In general, any subject having, or suspected of having, a condition or disorder, may be treated using the compositions and methods of the present disclosure. Subjects receiving treatment according to the methods described herein are mammalian subjects, and typically human patients. Other mammals that may be treated according to the present disclosure include companion animals such as dogs and cats, farm animals such as cows, horses, and swine, as well as birds and more exotic animals (e.g., those found in zoos or nature preserves).

In accordance with one aspect of the present disclosure, methods are described herein for treating tissue damage resulting from a cancer treatment (e.g., radiation therapy or chemotherapy) delivered to a subject in need thereof. In accordance with another aspect of the present disclosure, methods are described herein for treating a human patient for tissue damage resulting from exposure to radiation. Thus, in various embodiments for example, the exposure to radiation in various embodiments may be an accidental radiation exposure, an unintentional radiation exposure, or an intentional radiation exposure. As noted above, treatment of tissue damage as described herein may include both inhibition (i.e., prophylaxis) and amelioration of any tissue damage that may result from an occurrence or activity. In general, the methods involve administering to the subject a therapeutically effective amount of the pentaaza macrocyclic ring complex. In one preferred embodiment, the complex is the dichloro complex form of Formula (GC4419), although other pentaaza macrocyclic ring complexes as described herein may also be used.

Treatment of tissue damage resulting from a cancer treatment or other radiation exposure in accordance with the methods described herein involves the administration of a therapeutically effective amount of the pentaaza macrocyclic ring complex, such as but not limited to, GC4419. In general, a range of therapeutically effective amounts may be used, depending, for example, on the compound selected and its safety and efficacy, the type, location, and severity of the tissue damage, among other factors. Examples of tissue damage that may be treated can include oral mucositis and other forms of tissue damage, including tissue damage affecting the mucosal lining of the upper and lower gastrointestinal tract.

According to yet another embodiment, the formulation can be used for treatment of cancers and/or tumors. Cancer and tumors generally refer to or describe the physiological condition in mammals that is typically characterized by unregulated cell growth. By means of the pharmaceutical formulations herein, various tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver.

In one embodiment, the tumor or cancer is chosen from adenoma, angio-sarcoma, astrocytoma, epithelial carcinoma, germinoma, glioblastoma, glioma, hamartoma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

Thus, for example, the present disclosure provides methods for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma.

For example, particular leukemias that can be treated with the formulations and methods described herein include, but are not limited to, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas can also be treated with the formulations and methods described herein. Lymphomas are generally neoplastic transformations of cells that reside primarily in lymphoid tissue. Lymphomas are tumors of the immune system and generally are present as both T cell- and as B cell-associated disease. Among lymphomas, there are two major distinct groups: non-Hodgkin's lymphoma (NHL) and Hodgkin's disease. Bone marrow, lymph nodes, spleen and circulating cells, among others, may be involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then re-infused in order to repopulate the patient's hematopoietic system.

Other hematological malignancies that can be treated with the combinations and methods described herein include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

In one embodiment, the methods and formulations described herein are used to treat a cancer that is any of breast cancer, melanoma, oral squamous cell carcinoma, lung cancer including non-small cell lung cancer, renal cell carcinoma, colorectal cancer, prostate cancer, brain cancer, spindle cell carcinoma, urothelial cancer, bladder cancer, colorectal cancer, head and neck cancers such as squamous cell carcinoma, and pancreatic cancer. In yet another embodiment, the methods and formulations described herein are used to treat a cancer that is any of head and neck cancer and lung cancer.

As noted above, the diseases or conditions treated in accordance with the methods described herein may be any disease or condition that is/are treatable with the pentaaza macrocyclic ring complex. In one embodiment, for example, the disease or condition is selected from cancer, a cardiovascular disorder, a cerebrovascular disorder, a dermatological disorder, a fibrotic disorder, a gastrointestinal disorder, an immunological disorder, an inflammatory disorder, a metabolic disorder, a neurological disorder, an ophthalmic disorder, a pulmonary disorder, an infectious disease, and combinations thereof. By way of example, uses include the treatment of inflammatory and hyperproliferative skin diseases and cutaneous manifestations of immunologically-mediated illnesses, such as psoriasis, atopic dermatitis, contact dermatitis and further eczematous dermatitises, seborrhoeis dermatitis, lichen planus, pemphigus, bullous pemphigoid, epidermolysis bullosa, urticaria, angioedemas, vasculitides, erythemas, cutaneous eosinophilias, lupus erythematosus, acne and alopecia greats; various eye diseases (autoimmune and otherwise) such as keratoconjunctivitis, vernal conjunctivitis, uveitis associated with Behcet's disease, keratitis, herpetic keratitis, conical cornea, dystrophia epithelialis corneae, corneal leukoma, and ocular pemphigus. In addition, reversible obstructive airway disease, which includes conditions such as asthma (for example, bronchial asthma, allergic asthma, intrinsic asthma, extrinsic asthma and dust asthma), particularly chronic or inveterate asthma (for example, late asthma and airway hyper-responsiveness), bronchitis, allergic rhinitis, and the like, can be treated, prevented, and/or ameliorated in accordance with the methods described herein. Other treatable diseases and conditions include inflammation of mucosa and blood vessels such as gastric ulcers, vascular damage caused by ischemic diseases and thrombosis. Moreover, hyperproliferative vascular diseases such as intimal smooth muscle cell hyperplasia, restenosis and vascular occlusion, particularly following biologically- or mechanically-mediated vascular injury, could be treated by the compounds described herein.

Still other treatable diseases and conditions include, but are not limited to, cardiac diseases such as post myocardial infarction, pulmonary diseases such as pulmonary muscle changes or remodeling and chronic obstructive pulmonary disease (COPD); ischemic bowel diseases, inflammatory bowel diseases, necrotizing enterocolitis, intestinal inflammations/allergies such as Coeliac diseases, proctitis, eosinophilic gastroenteritis, mastocytosis, Crohn's disease and ulcerative colitis; nervous diseases such as multiple myositis, Guillain-Barre syndrome, Meniere's disease, polyneuritis, multiple neuritis, mononeuritis and radiculopathy; septic shock and related refractory hypotension; endocrine diseases such as hyperthyroidism and Basedow's disease; arthritis (for example rheumatoid arthritis, arthritis chronica progrediente and arthritis deformans) and rheumatic diseases; hematic diseases such as pure red cell aplasia, aplastic anemia, hypoplastic anemia, idiopathic thrombocytopenic purpura, autoimmune hemolytic anemia, agranulocytosis, pernicious anemia, megaloblastic anemia and anerythroplasia; bone diseases such as osteoporosis; respiratory diseases such as sarcoidosis, fibroid lung and idiopathic interstitial pneumonia; skin disease such as dermatomyositis, leukoderma vulgaris, ichthyosis vulgaris, photoallergic sensitivity and cutaneous T cell lymphoma; circulatory diseases such as arteriosclerosis, atherosclerosis, aortitis syndrome, polyarteritis nodosa and myocardosis; collagen diseases such as scleroderma, Wegener's granuloma and Sjogren's syndrome; adiposis; eosinophilic fasciitis; periodontal disease such as lesions of gingiva, periodontium, alveolar bone and substantia ossea dentis; nephrotic syndrome such as glomerulonephritis; male pattern aleopecia or alopecia senilis by preventing epilation or providing hair germination and/or promoting hair generation and hair growth; muscular dystrophy; Pyoderma and Sezary's syndrome; Addison's disease; active oxygen-mediated diseases, as for example organ injury such as ischemia-reperfusion injury of organs (such as heart, liver, kidney and digestive tract) which occurs upon preservation, transplantation, organ failure (single or multi-), or ischemic disease (for example, thrombosis and cardiac infarction); dyskinetic disorders such as Parkinson's disease, neuroleptic-induced parkinsonism and tardive dyskinesias; intestinal diseases such as endotoxin-shock, pseudomembranous colitis and colitis caused by drug or radiation; renal diseases such as ischemic acute renal insufficiency and chronic renal insufficiency; pulmonary diseases such as toxinosis caused by lung-oxygen or drug (for example, paracort and bleomycins), lung cancer and pulmonary emphysema; ocular diseases such as cataracta, siderosis, retinitis, pigmentosa, senile macular degeneration, vitreal scarring and corneal alkali burn; dermatitis such as erythema multiforme, linear IgA ballous dermatitis and cement dermatitis; and others such as gingivitis, periodontitis, sepsis, pancreatitis, diseases caused by environmental pollution (for example, air pollution), aging, carcinogenesis, metastasis of carcinoma and hypobaropathy; diseases caused by histamine or leukotriene-C4 release; Behcet's disease such as intestinal-, vasculo- or neuro-Behcet's disease, and also Behcet's which affects the oral cavity, skin, eye, vulva, articulation, epididymis, lung, kidney and so on. Furthermore, the compounds of the invention are useful for the treatment and prevention of hepatic disease such as immunogenic diseases (for example, chronic autoimmune liver diseases such as autoimmune hepatitis, primary biliary cirrhosis and sclerosing cholangitis), partial liver resection, acute liver necrosis (e.g., necrosis caused by toxin, viral hepatitis, shock or anoxia), B-virus hepatitis, non-A/non-B hepatitis, cirrhosis (such as alcoholic cirrhosis) and hepatic failure such as fulminant hepatic failure, late-onset hepatic failure and “acute-on-chronic” liver failure (acute liver failure on chronic liver diseases), and for treatment of bacterial or viral infections such as influenza or HIV infection, and moreover are useful for various diseases because of their useful activity such as augmentation of chemotherapeutic effect, cytomegalovirus infection, particularly HCMV infection, anti-inflammatory activity, sclerosing and fibrotic diseases such as nephrosis, scleroderma, fibrosis (e.g., pulmonary fibrosis and lung fibrosis, including cryptogenic fibrosing alveolitis, idiopathic interstitial pneumonias, ideopathic pulmonary fibrosis, ideopathic mediastinal fibrosis, fibrosis complicating anti-neoplastic therapy, radiation therapy, and chronic infection, including tuberculosis and aspergillosis and other fungal infections), arteriosclerosis, congestive heart failure, ventricular hypertrophy, post-surgical adhesions and scarring, stroke, myocardial infarction and injury associated with ischemia and reperfusion, and the like.

### Pharmaceutical Formulations

According to certain embodiments, the aqueous solutions may be administered via a parenteral route (e.g., intravenous, intraarterial, subcutaneous, rectal, subcutaneous, intramuscular, intraorbital, intracapsular, intraspinal, intraperitoneal, or intrasternal). However, other routes of administration may also be possible therewith, such as oral, topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable additives and/or excipients for use in combination with the compositions of the present disclosure are well known to those of ordinary skill in the art and are selected based upon a number of factors: the particular compound(s) and agent(s) used, and its/their concentration, stability and intended bioavailability; the subject, its age, size and general condition; and the route of administration. Pharmaceutically acceptable additives for use in the pharmaceutical compositions described herein are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), and A. J. Spiegel et al., Use of Nonaqueous Solvents in Parenteral Products, Journal of Pharmaceutical Sciences, Vol. 52, No. 10, pp. 917-927 (1963).

Formulations for certain pentaaza macrocyclic ring complexes are also described in, for example, in U.S. Pat. Nos. 5,610,293, 5,637,578, 5,874,421, 5,976,498, 6,084,093, 6,180,620, 6,204,259, 6,214,817, 6,245,758, 6,395,725, and 6,525,041 (each of which is hereby incorporated herein by reference in its entirety).

The above-described pharmaceutical compositions including the pentaaza macrocyclic compound may additionally include one or more additional pharmaceutically active components. Suitable pharmaceutically active agents that may be included in the compositions according to aspects of the present invention include, for instance, antiemetics, anesthetics, antihypertensives, antianxiety agents, anticlotting agents, anticonvulsants, blood glucose-lowering agents, decongestants, antihistamines, antitussives, antineoplastics, beta blockers, anti-inflammatory agents, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism agents, anti-Alzheimer's Disease agents, antibiotics, anti-depressants, and antiviral agents. The individual components of such combinations may be administered either sequentially or simultaneously in separate or combined pharmaceutical formulations.

In yet another embodiment, a kit may be provided that includes the pentaaza macrocyclic ring complex, for treatment of a condition. For example, the kit may comprise a first vessel or container having therein a formulation comprising the pentaaza macrocyclic ring complex in an aqueous solution, such as an oral or injectable formulation of the pentaaza macrocyclic ring complex. The kit may further comprise a label or other instructions for administration of the active agents, recommended dosage amounts, durations and administration regimens, warnings, listing of possible drug-drug interactions, and other relevant instructions, such as a label instructing therapeutic regimens (e.g., dosing, frequency of dosing, etc.) corresponding to any of those described herein.

### Combination Treatment with Cancer Therapy

In one embodiment, the aqueous solution comprising the pentaaza macrocyclic ring complex can be administered in combination with another cancer therapy, to provide therapeutic treatment. For example, the pentaaza macrocyclic ring complex may be administered as a part of a radiation therapy or chemotherapy regimen.

In general, the temporal aspects of the administration of the pentaaza macrocyclic ring complex may depend for example, on the particular radiation therapy that is selected, or the type, nature, and/or duration of the radiation exposure. Other considerations may include the disease or disorder being treated and the severity of the disease or disorder; activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors. For example, the compound may be administered in various embodiments before, during, and/or after the administration of the radiation therapy (e.g., before, during or after exposure to and/or before, during or after a course of radiation therapy comprising multiple exposures and/or doses). By way of another example, the compound may be administered in various embodiments before, during, and/or after an exposure to radiation. If desired, the effective dose can be divided into multiple doses for purposes of administration; consequently, single dose compositions may contain such amounts or submultiples thereof to make up the dose.

In one embodiment, for example, the pentaaza macrocyclic ring complex can be are administered to the patient prior to or simultaneous with the radiation exposure and/or chemotherapy dose. In another embodiment, for example, the compound is administered to the patient prior to, but not after, the radiation exposure and/or chemotherapy dose. In yet another embodiment, the pentaaza macrocyclic ring complex is administered to the patient at least 15 minutes, 30 minutes, 45 minutes, 60 minutes, 90 minutes, 180 minutes, 0.5 days, 1 day, 3 days, 5 days, one week, two weeks, three weeks, four weeks, five weeks, six weeks, seven weeks, eight weeks, nine weeks, ten weeks, eleven weeks, twelve weeks, or longer, prior to the radiation exposure and/or chemotherapy dose, such as an initial radiation exposure in a course of radiation treatment, or prior to another dose or dose fraction of radiation that is one of the doses or dose fractions of radiation in the course of treatment. In still other embodiments, for example, the pentaaza macrocyclic ring complex is administered to the patient after the radiation exposure and/or chemotherapy dose; thus, for example, the compound may be administered up to 15 minutes, 30 minutes, 45 minutes, 60 minutes, 90 minutes, or 180 minutes, 0.5 days, 1 day, 3 days, 5 days, one week, two weeks, three weeks, four weeks, five weeks, six weeks, seven weeks, eight weeks, nine weeks, ten weeks, eleven weeks, twelve weeks, or longer, after the radiation exposure, which may be a dose or dose fraction of radiation in a multi-dose course of radiation therapy, or may be the single or final dose or dose fraction of radiation in the radiation therapy, or after a chemotherapy dose.

In one embodiment, a course of radiation therapy includes a plurality of radiation doses or dose fractions given over a predetermined period of time, such as over the course of hours, weeks, days and even months, with the plural doses or dose fractions being either of the same magnitude or varying. That is, a course of radiation therapy can comprise the administration of a series of multiple doses or dose fractions of radiation. In one embodiment, the pentaaza macrocyclic ring complex can be administered before one or more radiation doses or dose fractions in the series, such as before each radiation dose or dose fraction, or before some number of the radiation doses or dose fractions. Furthermore, the administration of the pentaaza macrocyclic ring complex during the course of radiation therapy can be selected to enhance the cancer treating effects of the radiation therapy. In one embodiment, the pentaaza macrocyclic ring complex is administered within a predetermined duration before or after of each dose or dose fraction, such as the predetermined duration discussed above. In another embodiment, the pentaaza macrocyclic ring complex is administered within a predetermined duration of time before or after only select doses or dose fractions.

A suitable overall dose to provide during a course of therapy can be determined according to the type of treatment to be provided, the physical characteristics of the patient and other factors, and the dose fractions that are to be provided can be similarly determined. In one embodiment, a dose fraction of radiation that is administered to a patient may be at least 1.8 Gy, such as at least 2 Gy, and even at least 3 Gy, such as at least 5 Gy, and even at least 6 Gy. In yet another embodiment, a dose fraction of radiation that is administered to a patient may be at least 10 Gy, such as at least 12 Gy, and even at least 15 Gy, such as at least 18 Gy, and even at least 20 Gy, such as at least 24 Gy. In general, a dose fraction of radiation administered to a patient will not exceed 54 Gy. A total dose administered during a course of therapy (i.e. a sum of all dose fractions) may be at least 20 Gy, at least 30 Gy, at least 40 Gy, at least 50 Gy, at least 60 Gy, and/or at least 70 Gy. For example, a total dose may be in a range of 50 Gy to 75 Gy, such as in a range of 60 Gy to 72 Gy. Furthermore, it should be noted that, in one embodiment, a dose fraction delivered to a subject may refer to an amount delivered to a specific target region of a subject, such as a target region of a tumor, whereas other regions of the tumor or surrounding tissue may be exposed to more or less radiation than that specified by the nominal dose fraction amount.

For example, in one embodiment, the overall dose of radiation provided during the course of therapy may be provided via a hypofractionation radiotherapy scheme, which typically involves providing relatively high dose fractions administered over relatively fewer sessions, as compared to lower dose fraction schemes. Examples of such hypofractionation radiotherapy methods can include, but are not limited to, stereotactic radiosurgery (SRS), which typically refers to a single-fraction treatment directed to targets such as intracranial and spinal targets, as well as stereotactic body radiation therapy (SBRT), which typically refers to multifractional treatment of targets such as intracranial and spinal targets, and also extracranial targets such as lung, liver, head and neck, pancreas and prostate. As an example, in one embodiment of a hypofractionation radiotherapy scheme, the overall dose of radiation provided during the course of therapy may be divided into less than 10 fractions, such as less than 8 fractions, less than 6 fractions, less than 5 fractions, less than 4 fractions, less than 3 fractions, less than 2 fractions and may even be provided in just one administration (single fraction). For example, in one embodiment, the overall dose of radiation provided during the course of therapy may be divided into from 1 to 10 fractions, such as from 1 to 6 fractions, and even from 1 to 5 fractions, such as from 2 to 5 fractions or even 2 to 4 fractions. As yet another example, the hypofractionation radiotherapy scheme can comprise dividing the overall dose of radiation provided during the course of therapy into dose fractions that are at least 10% ( 1/10) of the overall dose provided during therapy, such as at least 12.5% (⅛) of the overall dose, at least 16% (˜⅙) of the overall dose, at least 20% (⅕) of the overall dose, at least 25% (¼) of the overall dose, at least 30% (⅓) of the overall dose, at least 50% of the overall dose, and/or at least 100% of the overall dose may be provided in a single administration (single fraction). For example, in one embodiment, the overall dose of radiation provided during the course of therapy may be divided into fractions that provide from 10% to 100% of the overall dose in each fraction, such as from 16% to 100% of the overall dose, and even from 20% to 100% of the overall dose, such as from 20% to 50% of the overall dose or even from 25% to 50% of the overall dose. For example a dose fraction size may be at least 5 Gy, such as at least 6 Gy, at least 8 Gy, at least 10 Gy, at least 12 Gy, and even at least 15 Gy, such as at least 18 Gy, and even at least 20 Gy, such as at least 24 Gy, and typically do not exceed 54 Gy, such as less than 40 Gy and even less than 30 Gy. In one embodiment, dose fraction sizes may be in the range of from 5 Gy to 30 Gy, such as from 6 Gy to 28 Gy, and even from 8 Gy to 25 Gy. Furthermore, in one embodiment, the dose fractions may be administered no more than three times per day, and even no more than twice per day, such as no more than once per day, on consecutive or non-consecutive days and/or some combination thereof, and may be administered over a period of a few days and up to a few weeks, such as over a period of 1 to 15 days, 1 to 12 days, 1 to 10 days, 1 to 5 days, and even 1 to 3 days. Typically, the dose fractions making up the overall course of therapy will be administered in no more than 20 days, no more than 15 days, no more than 10 days, no more than 5 days, and even no more than 3 days.

As yet another example, in one embodiment, the overall dose of radiation provided during the course of therapy may be provided via a radiotherapy scheme that provides relatively lower dose fractions administered over relatively more sessions, as compared to, e.g., hypofractionation schemes. Examples of such lower dose fraction radiotherapy methods can include, but are not limited to, intensity-modulated radiation therapy (IMRT) and image guided radiation therapy (IGRT), which typically involve three-dimensional conformal therapy (3D-CRT) to match the administered radiation to a target volume. As an example, in one embodiment of such a radiotherapy scheme, the overall dose of radiation provided during the course of therapy may be divided into at least 15 fractions, such as at least 18 fractions, at least 20 fractions, at least 22 fractions, at least 25 fractions, at least 28 fractions, at least 30 fractions, at least 32 fractions, at least 35 fractions, and even at least 38 fractions, although the total number of fractions will typically be less than 50, such as less than 45, and even less than 42. For example, in one embodiment, the overall dose of radiation provided during the course of therapy may be divided into from 15 to 38 fractions, such as from 20 to 38 fractions, and even from 20 to 35 fractions, such as from 25 to 35 fractions. As yet another example, the radiotherapy scheme can comprise dividing the overall dose of radiation provided during the course of therapy into dose fractions that are no more than 7% ( 1/15) of the overall dose provided during therapy, such as no more than 6% ( 1/18) of the overall dose, no more than 5% ( 1/20) of the overall dose, no more than 4.5% ( 1/22) of the overall dose, no more than 4% ( 1/25) of the overall dose, no more than 3.6% ( 1/28) of the overall dose, no more than 3.3% ( 1/30) of the overall dose, no more than 3.1% ( 1/32) of the overall dose, no more than 2.8% of the overall dose ( 1/35), and even no more than 2.6% ( 1/38) of the overall dose. For example, in one embodiment, the overall dose of radiation provided during the course of therapy may be divided into fractions that provide from 2.5% to 8% of the overall dose in each fraction, such as from 2.8% to 5% of the overall dose, and even from 2.8% to 4% of the overall dose. For example a dose fraction size may be less than 5 Gy, such as less than 4 Gy, less than 3.5 Gy, less than 3 Gy, less than 2.8 Gy, and even less than 2.5 Gy, such as less than 2.3 Gy, and even less than 2 Gy, such as less than 1.8 Gy, and typically is at least 0.5 Gy, such as at least 1 Gy and even at least 1.5 Gy. In one embodiment, dose fraction sizes may be in the range of from 1.5 Gy to 4.5 Gy, such as from 1.8 Gy to 3 Gy, and even from 2 Gy to 2.5 Gy. Furthermore, in one embodiment, the dose fractions may be administered no more than three times per day, and even no more than twice per day, such as no more than once per day, on consecutive or non-consecutive days, and/or a combination thereof (e.g., on consecutive weekdays), and in some embodiments may be administered over a period of a few days to a few weeks and even a few months, such as over a period of up to 3 weeks, up to 5 weeks, up to 6 weeks, up to 8 weeks and even up to 10 weeks, such as in a range of from 3 weeks to 10 weeks, or even in a range of from 5 weeks to 8 weeks. For example, the dose fractions making up the overall course of therapy can be administered in no more than 12 weeks, such as no more than 10 weeks and even no more than 8 weeks.

In yet another embodiment, the overall dose of radiation provided by the radiation scheme, whether in a relatively high dose fraction scheme or relatively low dose fraction scheme such as those described above, or other scheme, is selected to provide suitable treatment of the cancer. The overall dose may also be provided according to the specific dose fractionation scheme being administered, along with other factors. For example, in certain embodiments, a relatively larger overall dose may be administered as relatively smaller individual dose fractions. In one embodiment, the overall dose provided over the course of the therapy (i.e., the sum of the administered dose fractions), is at least 50 Gy, such as at least 55 Gy, at least 58 Gy, at least 60 Gy, at least 65 Gy, at least 68 Gy, at least 70 Gy, at least 72 Gy, and even at least 75 Gy. In certain embodiments, the overall dose does not exceed 80 Gy, such as not exceeding 78 Gy and even not exceeding 75 Gy. For example, the overall dose may be in a range of from 50 Gy to 75 Gy, such as from 55 Gy to 75 Gy, and even from 60 Gy to 70 Gy.

In yet another embodiment, the pentaaza macrocyclic ring complex can be administered as a part of a course of therapy that includes administration of a chemotherapeutic agent, such as for example a platinum-based chemotherapeutic agent (e.g., cisplatin). In chemotherapy, chemotherapeutic agents are administered to a patient to kill or control the growth of cancerous cells. A typical course of chemotherapy may include one or a plurality of doses of one or more chemotherapeutic agents, which can be administered over the course of days, weeks and even months. Chemotherapeutic agents can include at least one of: alkylating antineoplastic agents such as nitrogen mustards (e.g. cyclophosphamide, chlorambucil), nitrosoureas (e.g. n-nitroso-n-methylurea, carmustine, semustine), tetrazines (e.g. dacarbazine, mitozolimide), aziridines (e.g. thiotepa, mytomycin); anti-metabolites such as anti-folates (e.g. methotrexate and pemetrexed), fluoropyrimidines (e.g., fluorouracil, capecitabine), anthracyclines (e.g. doxorubicin, daunorubicin, epirubicin), deoxynucleoside analogs (e.g. cytarabine, gemcitabine, decitabine) and thiopurines (e.g., thioguanine, mercaptopurine); anti microtubule agents such as taxanes (e.g. paclitaxel, docetaxel); topoisomerase inhibitors (e.g. etoposide, doxorubicin, mitoxantrone, teniposide); antitumor antibiotics (e.g. bleomycin, mitomycin); and platins (e.g., cisplatin, carboplatin, oxaliplatin). For example, the chemotherapeutic agent may be selected from the group consisting of all-trans retinoic acid, arsenic trioxide, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, mechlorethamine, mercaptopurine, methotrexate, mitoxantrone, oxaliplatin, paclitaxel, pemetrexed, teniposide, tiguanine, valrubicin, vinblastine, vincristine, vindesine, and vinorelbine. The administration of many of the chemotherapeutic agents is described in the “Physicians' Desk Reference” (PDR), e.g., 1996 edition (Medical Economics Company, Montvale, N.J. 07645-1742, USA).

In one embodiment, the chemotherapeutic agent comprises a platinum-based anticancer agent, such as any selected from the group consisting of cisplatin, carboplatin, oxaliplatin, nedaplatin, lobaplatin, heptaplatin, dicycloplation, lipoplatin, LA-12 ((OC-6-43)-bis(acetato)(1-adamantylamine)amminedichloroplatinum (IV)), phosphaplatin, phenanthriplatin, ProLindac (AP5346), triplatin tetranitrate, picoplatin, satraplatin, pyriplatin and/or a pharmaceutically acceptable salt thereof. Examples of suitable doses of a platinum-based anticancer agent may be in a range from 10 mg/m² to 200 mg/m², such as 20 mg/m² to 100 mg/m². The dosing schedule of the platinum-based anti-cancer agent can similarly be selected according to the intended treatment and the platinum-based anti-cancer agent being provided. For example, in one embodiment, a suitable dosing schedule can comprise dosing a patient at a frequency of once or twice per day, two days, three days, four days, five days, six days, per week, per two weeks, per three weeks or per month.

According to yet another embodiment, a method of treatment can comprise a combination therapy of the pentaaza macrocyclic ring complex with an immunotherapeutic agent, such as an immune checkpoint inhibitor, an adoptive T-cell transfer therapy, and/or a cancer vaccine, which may be administered for the treatment of cancer, and may also optionally be administered as a part of a course of treatment also involving chemotherapeutic and/or radiation therapy.

### EXAMPLES

The following non-limiting examples are provided to further illustrate aspects of the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Effect of Order of Addition on Precipitate Formation in Aqueous Formulation

### Example 1

In this example, aqueous formulations were formed by combining a manganese-containing coordination complex corresponding to the structural formula of GC4419 disclosed herein, with sodium chloride and sodium bicarbonate (as a buffering agent), according to different orders of addition of these components, and the formulations were monitored to visually observe whether particles (precipitate) formed therein.

Specifically, a first set of formulations (Formulations A-1 and A-2) were prepared by (1) adding 9 mg/mL GC4419 to water (WFI) adjusted to a pH in a range of from 7.4-7.8, followed by (2) adding sodium chloride (0.9% NaCl), and finally adding (3) sodium bicarbonate (26 mM), with further water optionally added to obtain a desired concentration. A second set of formulations (Formulations B-1 and B-2) were prepared by adding 9 mg/mL GC4419 to water (WFI) adjusted to a pH in a range of from 7.4-7.8, followed by (2) sodium bicarbonate (26 mM), and finally (3) adding sodium chloride (0.9% NaCl), with further water optionally added to obtain a desired concentration. A third set of formulations (Formulations C-1, C-2 and C-3) were then prepared in the same way as Formulations A-1 and A-2. That is, the “A” Formulations and “C” Formulations differed from “B” Formulations in the order of addition of sodium bicarbonate with respect to sodium chloride added to the solution. Table 1 below shows the results in terms of observed visible particles (precipitate) for each of the formulations. Also shown are the results in terms of subvisible particles assayed as described in US Pharmacopeia General Chapter <788> and reported as numbers of particles >=10 μm and >=25 μm in diameter.

TABLE 1
Particulate Matter

| | Formulation | | | | | | |
|---|---|---|---|---|---|---|---|
| Time | A-1 | A-2 | B-1 | B-2 | C-1 | C-2 | C-3 |
| 0 | Visible: No | Visible: No | Visible: No | Visible: No | Visible: No | Visible: No | Visible: No |
| | Subvisible: | Subvisible: | Sub Visible: | Subvisible: | Subvisible: | Subvisible: | Subvisible: |
| | 224 ≥ 10 μm | 62 ≥ 10 μm | 499 ≥ 10 μm | 70 ≥ 10 μm | 98 ≥ 10 μm | 70 ≥ 10 μm | 104 ≥ 10 μm |
| | 27 ≥ 25 μm | 3 ≥ 25 μm | 1 ≥ 25 μm | 4 ≥ 25 μm | 11 ≥ 25 μm | 0 ≥ 25 μm | 49 ≥ 25 μm |
| 1 | Visible: No | Visible: No | Visible: No | Visible: No | Visible: No | Visible: No | Visible: No |
| | | | | | Subvisible: | Subvisible: | |
| | | | | | 92 ≥ 10 μm | 88 ≥ 10 μm | |
| | | | | | 2 ≥ 25 μm | 49 ≥ 25 μm | |
| 3 | Visible: No | Visible: No | Visible: No | Visible: No | Visible: No | Visible: No | Visible: No |
| | | | | | Subvisible: | | Subvisible: |
| | | | | | 16 ≥ 10 μm | | 28 ≥ 10 μm |
| | | | | | 1 ≥ 25 μm | | 0 ≥ 25 μm |
| 6 | Visible: No | Visible: No | Visible: No | Visible: No | Visible: No | | |
| | | | | | Subvisible: | | |
| | | | | | 56 ≥ 10 μm | | |
| | | | | | 1 ≥ 25 μm | | |
| 9 | Visible: * | Visible: No | Visible: Yes | Visible: Yes | | | |
| | | | Subvisible: | Subvisible: | | | |
| | | | 1527 ≥ 10 μm | 1652 ≥ 10 μm | | | |
| | | | 15 ≥ 25 μm | 13 ≥ 25 μm | | | |
| 12 | Visible: No | Visible: No | Visible: Yes | Visible: Yes | | | |
| | | | Subvisible: | Subvisible: | | | |
| | | | 1756 ≥ 10 μm | 1492 ≥ 10 μm | | | |
| | | | 15 ≥ 25 μm | 17 ≥ 25 μm | | | |

* No observation of visible particulate made at this time point

Accordingly, as can be seen from the above, the “A” and “C” Formulations in which sodium chloride was added before sodium bicarbonate resulted in no observable visible precipitate even after 9 months and 12 months, and much lower numbers of subvisible particles. However, by merely reversing the order of addition, the “B” Formulations in which sodium bicarbonate was added before sodium chloride resulted in observable precipitate and much higher numbers of subvisible particles after 9 months, even though such precipitate was not observable immediately upon manufacture of the formulation (e.g. at 0 months).

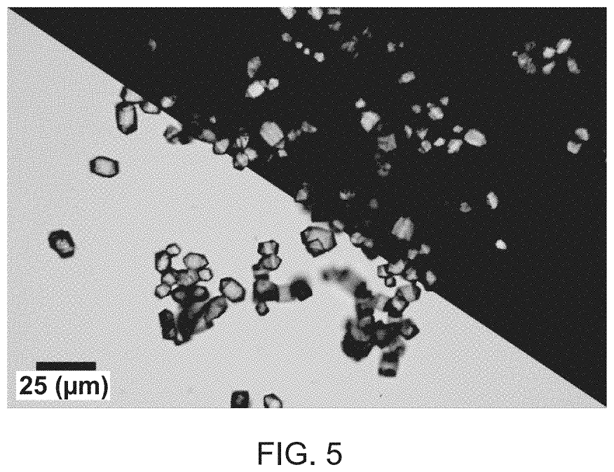

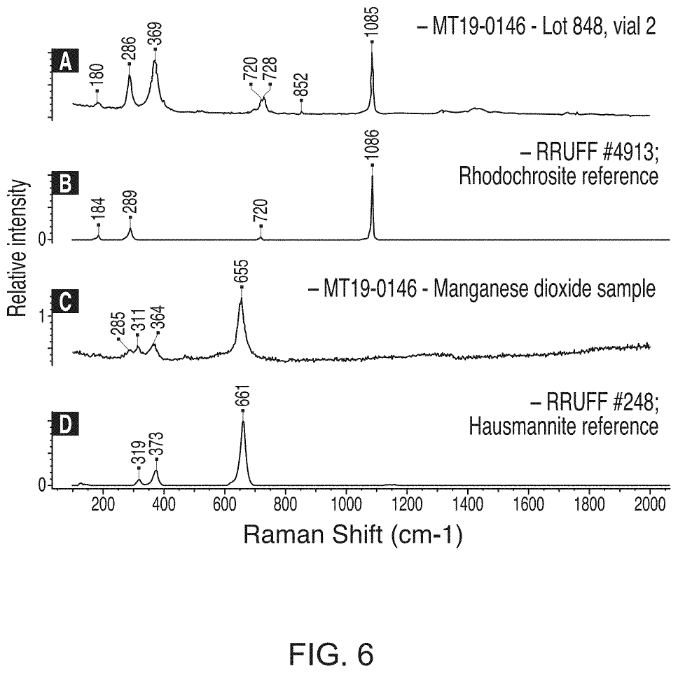

The precipitate formed after 9 months in the “B” Formulations was further subjected to analysis to determine the chemical composition and physical characteristics of the precipitate, including by Polarized Light Microscopy (PLM), Elemental Analysis using a field emission scanning electron microscope (FESEM) coupled to an energy-dispersive X-ray spectrometer (EDS), Electron Backscatter Diffraction (EBSD), X-Ray Fluorescence (XRF), and Raman Microspectroscopy. Analysis by microscopy determined the precipitate to contain crystals consistent in appearance with rhodochrosite (manganese carbonate, MnCO₃), and analysis of these crystals by the Raman Microspectroscopy confirmed this identification. FIG. 5 is a photo showing the MnCO₃ crystals obtained from the “B” Formulations, as appearing in plain polarized light (lower left) and between crossed polars (upper right). FIG. 6 shows the Raman spectra for the MnCO₃ crystals obtained from the “B” formulations (top spectrum—A), as compared to a library reference spectrum for rhodochrosite (2nd spectrum—B), a Raman spectrum collected from a sample of MnO₂ (3^{rd} spectrum—C), and a library reference spectrum of Hausmannite, Mn₃O₄ (bottom spectrum—D).

Furthermore, formulations prepared in the same manner as the “A” and “C” Formulations, but in concentrations of 3 mg/mL and 10 mg/mL GC4419 (instead of 9 mg/mL in A-1, A-2, C-1, C-2 and C-3 above), also did not exhibit formation of precipitate upon visual inspection at the 9 month or 12 month date following preparation thereof.

### Example 2

In this example, aqueous formulations were formed by combining MnCl₂, with sodium chloride and sodium bicarbonate (as a buffering agent), according to different orders of addition of these components, to assess the effects of order of addition on the precipitation of manganese from solution over time.

Specifically, a first set of formulations (Formulation 1—“Order NaCl 1st”) were prepared by (1) adding 0.026, 0.26, 2.6 and 26 mM of MnCl₂ to water (WFI) adjusted to a pH in a range of from 7.4 to 7.8, followed by (2) adding sodium chloride (0.9% NaCl), and finally adding (3) sodium bicarbonate (26 mM). A second set of formulations (Formulation 2—“Order NaHCO₃ 1st”) were prepared by adding 0.026, 0.26, 2.6 and 26 mM of MnCl₂ to water (WFI) adjusted to a pH in a range of from 7.4 to 7.8, followed by (2) sodium bicarbonate (26 mM), and finally (3) adding sodium chloride (0.9% NaCl). A third set of formulations (Formulation 3—“Vehicle”) were prepared by adding 0.026, 0.26, 2.6 and 26 mM of MnCl₂ to water (WFI) adjusted to a pH in a range of from 7.4 to 7.8, followed by adding (2) a combined solution of sodium bicarbonate (26 mM), and sodium chloride (0.9% NaCl). That is, Formulation 2 differed from Formulation 1 and Formulation 3 in that sodium bicarbonate was added before addition of sodium chloride in the Formulation 2, as opposed to adding simultaneously (Formulation 3) or after addition of sodium chloride (Formulation 1).

The amount of manganese precipitate produced at 1 day and at 6 days following manufacture of the Formulations (1)-(3) were assessed by ICP-MS storage stability assay involving filtering the formulations through a 0.45 micrometer filter, washing the filter with pH 8.0 water, digesting the filter contents with nitric acid, and performing inductively coupled mass-spectrometry (ICP-MS) to detect manganese content of any precipitate.

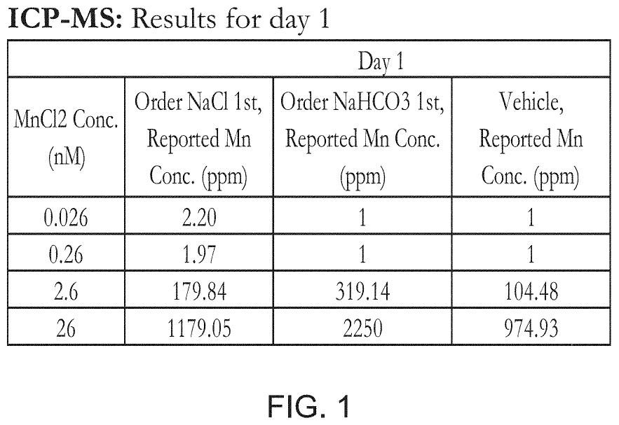

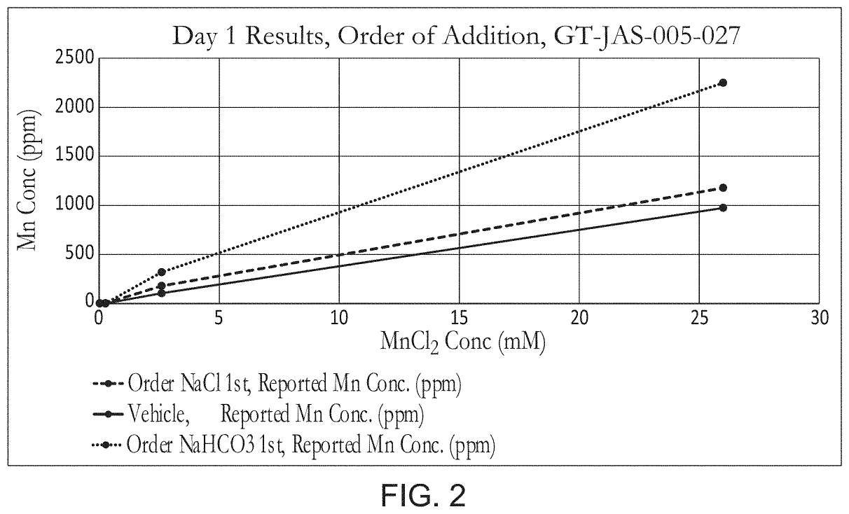

Referring to FIGS. 1-2, the results at day 1 following manufacture of the Formulations (1)-(3) can be seen. Specifically, it can be seen that while very low concentrations of MnCl₂ (0.026 ppm or 0.26 mM MnCl₂) resulting in very low or negligible amounts of manganese precipitate at 1 day (2.20 ppm or 1 ppm detected Mn), the Formulations (1)-(3) solutions having 2.6 mM or 26 mM MnCl₂ provided significantly different amounts of precipitate, depending on order of addition of the aqueous solution components. In particular, while Formulations (1) and (3) exhibited 179.84 ppm Mn and 104.48 ppm Mn for the 2.6 mM MnCl₂ solution, and 1179.05 ppm Mn and 974.93 ppm Mn for the 26 mM MnCl₂ solution at day 1, the Formulation (2) where sodium bicarbonate was added first exhibited an approximately 177% increase in measured Mn, of 319.14 ppm Mn for the 2.6 mM MnCl₂ solution, and 2250 ppm Mn for the 26 mM MnCl₂ solution at day 1. These results are displayed in chart form in FIG. 1, and graphically displayed in FIG. 2.

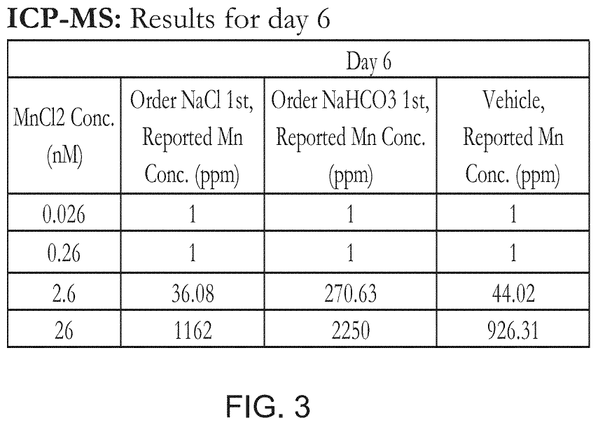

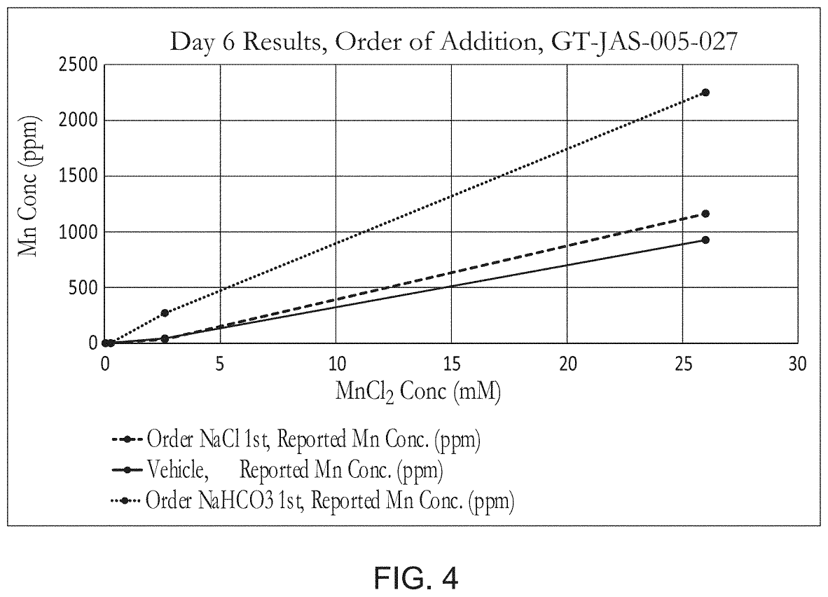

Similarly, Referring to FIGS. 3-4, the results at day 6 following manufacture of the Formulations (1)-(3) can be seen. As at day 1, it can be seen that very low concentrations of MnCl2 (0.026 ppm or 0.26 mM MnCl₂) resulted in very low or negligible amounts of manganese precipitate at 1 day (1 ppm detected Mn). However, the Formulation (1)-(3) solutions having 2.6 mM or 26 mM MnCl₂ provided further significant differences in the amounts of precipitate, depending on order of addition of the aqueous solution components. In particular, while Formulations (1) and (3) exhibited 36.08 ppm Mn and 44.02 ppm Mn for the 2.6 mM MnCl₂ solution, and 1162 ppm Mn and 926.31 ppm Mn for the 26 mM MnCl₂ solution at day 6, the Formulation (2) where sodium bicarbonate was added first exhibited an approximately 750% increase in measured Mn, of 270.63 ppm Mn for the 2.6 mM MnCl₂ solution, and 2250 ppm Mn for the 26 mM MnCl₂ solution at day 6. These results are displayed in chart form in FIG. 3, and graphically displayed in FIG. 4.

Accordingly, the results show that relatively small amounts of Mn-containing component in the aqueous formulation can result in the formation of significant amounts of precipitate in a case where sodium bicarbonate is added to the Mn-containing component prior to addition of sodium chloride. That is, the sodium chloride appears to provide a protective effect to reduce the formation of precipitate when added before or simultaneously with sodium bicarbonate to a solution with a Mn-containing component, so as to provide an excess of chloride ion as compared to the dianion generated by sodium bicarbonate.

What is claimed is:

1. A method of manufacturing an aqueous formulation of a manganese-containing coordination complex, the aqueous formulation comprising the manganese-containing coordination complex, a chloride anion, and a dianion, the method comprising:
combining a source of the manganese-containing coordination complex with a source of chloride anion in an aqueous solution; and
simultaneously with or following combination of the source of chloride anion and the source of manganese-containing coordination complex in the aqueous solution, providing a source of a dianion to the aqueous solution to form the aqueous formulation,
wherein an amount of the source of chloride anion that is combined with the manganese-containing coordination complex is sufficient to provide a concentration of chloride ion in the aqueous formulation that is in excess of a concentration of dianion in the aqueous formulation.

2. The method according to claim 1, wherein the manganese-containing coordination complex comprises manganese coordinated to a macrocyclic ligand.

3. The method according to any preceding claim, wherein the manganese-containing coordination complex comprises any one selected from the group consisting of a pentaaza macrocyclic ligand, a tetraaza macrocyclic ligand, a porphyrin macrocyclic ligand, a phthalocyanine macrocyclic ligand, and a crown ether macrocyclic ligand.

4. The method according to any preceding claim, wherein the manganese-containing coordination complex comprises manganese coordinated to one or more monodentate or polydentate ligands via nitrogen atoms of the one or more ligands.

5. The method according to any preceding claim, wherein the manganese-containing coordination complex comprises an Mn(II) coordination complex.

6. The method according to any preceding claim, wherein the source of manganese-containing coordination complex further comprises a Mn(II)-containing component that comprises one or more of Mn(II) in an uncoordinated state, or as coordinated to one or more ligands that are other than one or more ligands of the manganese-containing coordination complex.

7. The method according to claim 6, wherein the Mn(II)-containing component is present in the source of manganese-containing coordination complex in a ratio by weight of the Mn(II)-containing component to the manganese-containing coordination complex that is in a range of from 1:100,000 to 1:100, and/or a range of from 1:75,000 to 1:1,000, and/or a range of from 1:50,000 to 1:5,000, and/or a range of from 1:15,000 to 1:8,000.

8. The method according to any preceding claim, wherein the manganese-containing coordination complex comprises a pentaaza macrocyclic ring complex having a structure according to Formula (I) 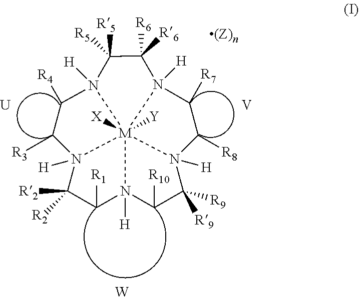
wherein
M is Mn²⁺ or Mn³⁺,
R₁, R₂, R′₂, R₃, R₄, R₅, R′₅, R₆, R′₆, R₇, R₈, R₉, R′₉, and R₁₀ are independently hydrogen, hydrocarbyl, substituted hydrocarbyl, heterocyclyl, an amino acid side chain moiety, or a moiety selected from the group consisting of —OR₁₁, —NR₁₁R₁₂, —COR₁₁, —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), and —OP(O)(OR₁₁)(OR₁₂), wherein R₁₁ and R₁₂ are independently hydrogen or alkyl;
U, together with the adjacent carbon atoms of the macrocycle, forms a fused substituted or unsubstituted, saturated, partially saturated or unsaturated, cycle or heterocycle having 3 to 20 ring carbon atoms;
V, together with the adjacent carbon atoms of the macrocycle, forms a fused substituted or unsubstituted, saturated, partially saturated or unsaturated, cycle or heterocycle having 3 to 20 ring carbon atoms;
W, together with the nitrogen of the macrocycle and the carbon atoms of the macrocycle to which it is attached, forms an aromatic or alicyclic, substituted or unsubstituted, saturated, partially saturated or unsaturated nitrogen-containing fused heterocycle having 2 to 20 ring carbon atoms, provided that when W is a fused aromatic heterocycle the hydrogen attached to the nitrogen which is both part of the heterocycle and the macrocycle and R₁ and R₁₀ attached to the carbon atoms which are both part of the heterocycle and the macrocycle are absent;
X and Y represent suitable ligands which are derived from any monodentate or polydentate coordinating ligand or ligand system or the corresponding anion thereof;
Z is a counterion;
n is an integer from 0 to 3; and
the dashed lines represent coordinating bonds between the nitrogen atoms of the macrocycle and the transition metal, manganese.

9. The method according to claim 8, wherein R₁, R₂, R′₂, R₃, R₄, R₅, R′₅, R₆, R′₆, R₇, R₈, R₉, R′₉, and R₁₀ are each hydrogen.

10. The method according to claim 8 or 9, wherein W is an unsubstituted pyridine moiety.

11. The method according to any one of claims 8-10, wherein U and V are transcyclohexanyl fused rings.

12. The method according to any preceding claim, wherein the manganese-containing coordination complex comprises a structure according to Formula (II): 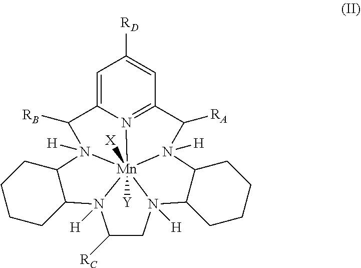
wherein
X and Y represent suitable ligands which are derived from any monodentate or polydentate coordinating ligand or ligand system or the corresponding anion thereof; and
R_{A}, R_{B}, R_{C}, and R_{D} are independently hydrogen, hydrocarbyl, substituted hydrocarbyl, heterocyclyl, an amino acid side chain moiety, or a moiety selected from the group consisting of —NR₁₁R₁₂, —COR₁₁, —CO₂R₁₁, —CONR₁₁R₁₂, —SR₁₁, —SOR₁₁, —SO₂R₁₁, —SO₂NR₁₁ R₁₂, —N(OR₁₁)(R₁₂), —P(O)(OR₁₁)(OR₁₂), —P(O)(OR₁₁)(R₁₂), and —OP(O)(OR₁₁)(OR₁₂), wherein R₁₁ and R₁₂ are independently hydrogen or alkyl.

13. The method according to any preceding claim, wherein the manganese-containing coordination complex is represented by Formula (III) or Formula (IV): 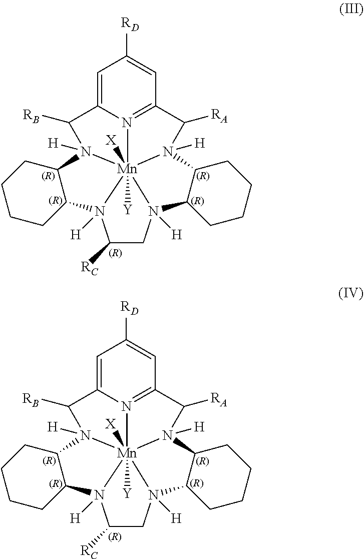
wherein
X and Y represent suitable ligands which are derived from any monodentate or polydentate coordinating ligand or ligand system or the corresponding anion thereof; and
R_{A}, R_{B}, R_{C}, and R_{D} are independently hydrogen, hydrocarbyl, substituted hydrocarbyl, heterocyclyl, an amino acid side chain moiety, or a moiety selected from the group consisting of —CO₂R₁₁, —CONR₁₁R₁₂, —SR₁₁, —SOR₁₁, —SO₂R₁₁, —SO₂NR₁₁R₁₂, —N(OR₁₁)(R₁₂), —P(O)(OR₁₁)(OR₁₂), —P(O)(OR₁₁)(R₁₂), and —OP(O)(OR₁₁)(OR₁₂), wherein R₁₁ and R₁₂ are independently hydrogen or alkyl.

14. The method according to any preceding claim, wherein the manganese-containing coordination complex is represented by a formula selected from the group consisting of Formulae (V)-(XVI):

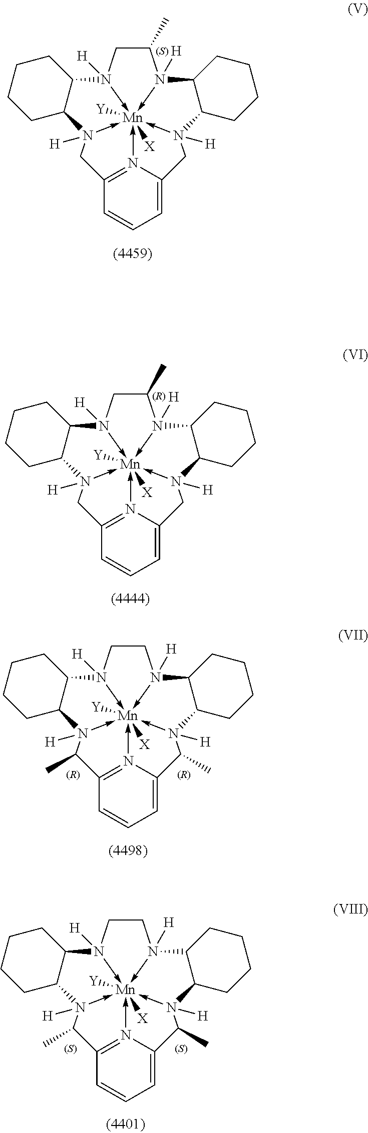

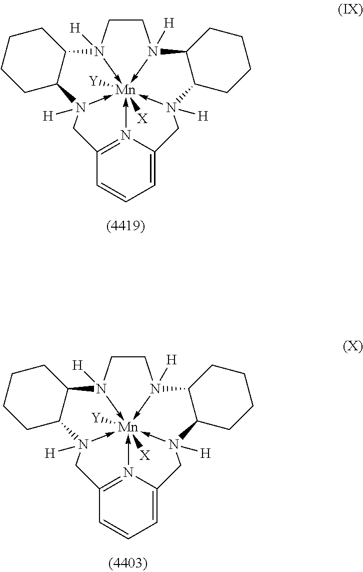

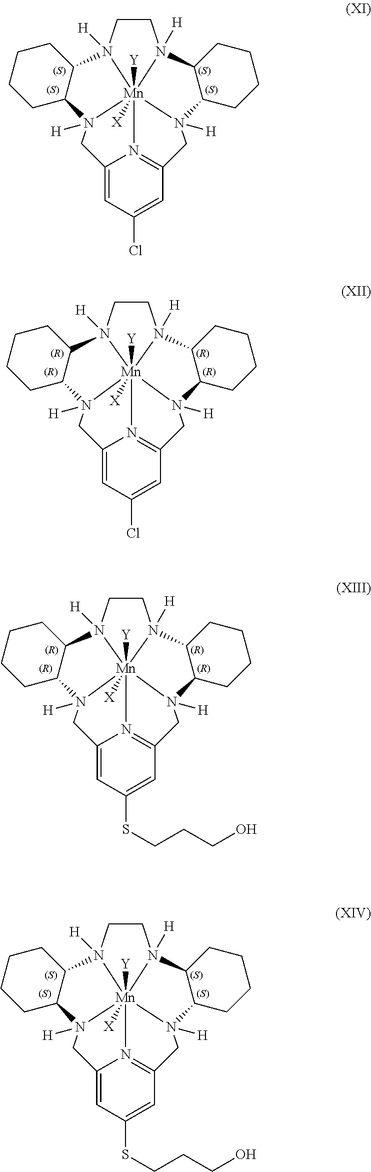

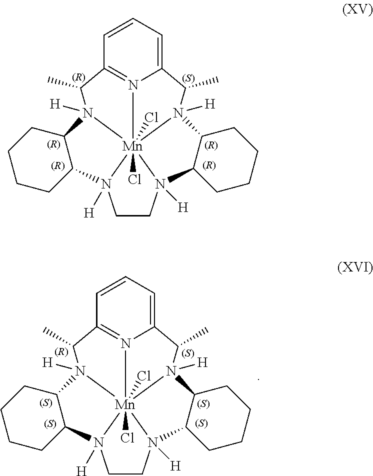

15. The method according to any of claims 8-14, wherein X and Y are independently selected from substituted or unsubstituted moieties of the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid, aryl carboxylic acid, urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate, aryl thiocarbamate, alkylaryl thiocarbamate, alkyl dithiocarbamate, aryl dithiocarbamate, alkylaryl dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins, or the corresponding anions thereof;
or X and Y correspond to —O—C(O)—X₁, where each X₁ is —C(X₂)(X₃)(X₄), and
each X₁ is independently substituted or unsubstituted phenyl or —C(—X₂)(—X₃)(—X₄);
each X₂ is independently substituted or unsubstituted phenyl, methyl, ethyl or propyl;
each X₃ is independently hydrogen, hydroxyl, methyl, ethyl, propyl, amino, —X₅C(═O)R₁₃ where X is NH or O, and R₁₃ is C1-C18 alkyl, substituted or unsubstituted aryl or C1-C18 aralkyl, or —OR₁₄, where R₁₄ is C1-C18 alkyl, substituted or unsubstituted aryl or C1-C18 aralkyl, or together with X₄ is (═O); and
each X₄ is independently hydrogen or together with X₃ is (═O);
or X and Y are independently selected from the group consisting of charge-neutralizing anions which are derived from any monodentate or polydentate coordinating ligand and a ligand system and the corresponding anion thereof;
or X and Y are independently attached to one or more of R₁, R₂, R′₂, R₃, R₄, R₅, R′₅, R₆, R′₆, R₇, R₈, R₉, R′₉, and R₁₀.

16. The method according to any one of claims 8-15, wherein X and Y are independently selected from the group consisting of fluoro, chloro, bromo, and iodo anions.

17. The method according to any one of claims 8-15, wherein X and Y are independently selected from the group consisting of alkyl carboxylates, aryl carboxylates and arylalkyl carboxylates.

18. The method according to any one of claims 8-15, wherein X and Y are independently amino acids.

19. The method according to any one of claims 1-16, wherein the manganese-containing coordination complex is a compound represented by the formula:

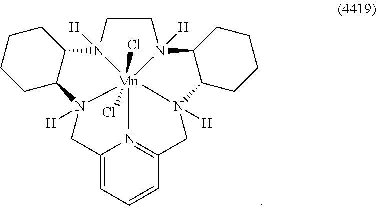

20. The method according to any one of claims 1-16, wherein the manganese-containing coordination complex is a compound represented by the formula:

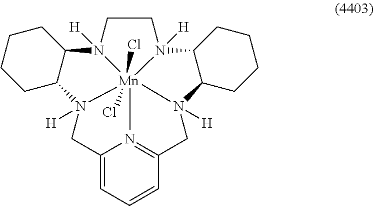

21. The method according to any one of claims 1-16, wherein the manganese-containing coordination complex is a compound represented by the formula:

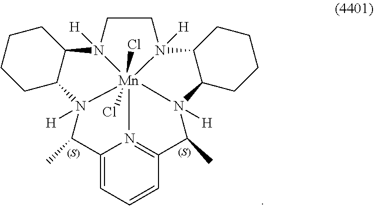

22. The method according to any one of claims 1-16, wherein the manganese-containing coordination complex is a compound represented by the formula:

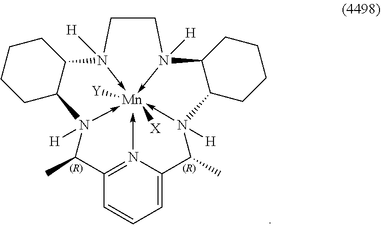

23. The method according to any any one of claims 1-16, wherein the manganese-containing coordination complex is a compound represented by the formula:

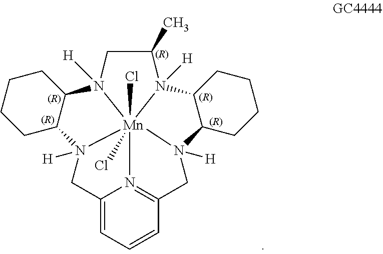

24. The method according to any one of claims 1-15 and 17-18, wherein the manganese-containing coordination complex is represented by the formula:

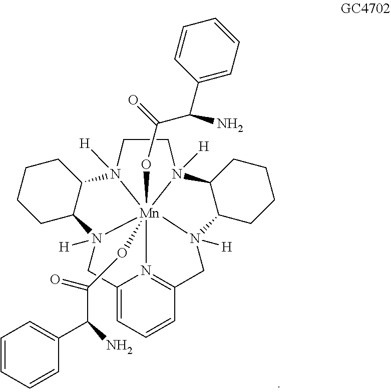

25. The method according to any one of claims 1-15 and 17, wherein the manganese-containing coordination complex is represented by the formula:

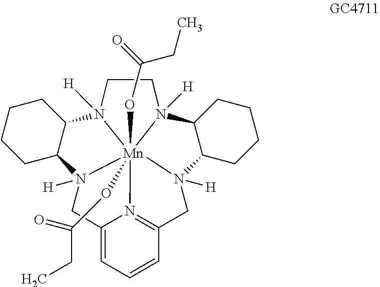

26. The method according to any one of claims 1-15 and 17, wherein the manganese-containing coordination complex is represented by the formula:

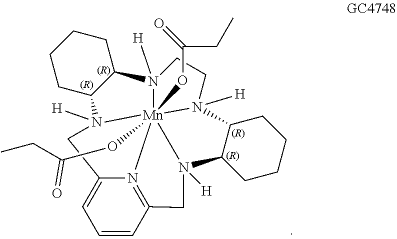

27. The method according to any preceding claim, wherein the source of chloride anion comprises a salt capable of forming chloride anions in aqueous solution.

28. The method according to any preceding claim, wherein the source of chloride anion comprises at least one selected from the group consisting of sodium chloride, potassium chloride, calcium chloride, and magnesium chloride.

29. The method according to any preceding claim, comprising adding an amount of the source of chloride anion that is sufficient to provide a concentration of chloride anion in the aqueous formulation of at least 100 mM, at least 110 mM, at least 115 mM, at least 120 mM, at least 130 mM, at least 145 mM and/or at least 150 mM, and no more than 1000 mM, no more than 200 mM, no more than 180 mM, no more than 175 mM, no more than 160 mM and/or no more than 155 mM.

30. The method according to any preceding claim, wherein the source of dianion comprises a bicarbonate salt and/or a phosphate salt.

31. The method according to any preceding claim, comprising adding an amount of the source of dianion that is sufficient to provide a concentration of the dianion in the aqueous formulation of at least 0.1 mM, at least 0.25 mM, at least 1 mM, and/or at least 2.5 mM, and no more than 26 mM, no more than 15 mM, and/or no more than 10 mM.

32. The method according to any preceding claim, wherein the source of dianion comprises a buffering agent, and wherein the source of dianion is adding in an amount sufficient to buffer the aqueous formulation within a pH range of from 7 to 10, and/or a pH range of from 7.5 to 9.

33. The method according to any preceding claim, wherein the concentration of chloride anion in the aqueous formulation exceeds the concentration of the dianion in the formulation by a ratio of the concentration in mol/L chloride anion to dianion of at least 10:1, at least 100:1, at least 250:1, at least 500:1, at least 750:1, at least 1000:1, at least 5000:1, and/or at least 10,000:1.

34. The method according to any preceding claim, wherein at least 75 mol %, at least 85 mol %, at least 90 mol %, at least 95 mol %, at least 98 mol %, at least 99 mol %, and/or the entire molar amount of the source of dianion added to form the aqueous formulation is added simultaneously with or following combination of the source of chloride anion with the manganese-containing coordination complex.

35. The method according to any preceding claim, wherein no amount of the source of dianion is combined with the manganese-containing coordination complex prior to combining the manganese-containing coordination complex and source of chloride anion.

36. The method according to any preceding claim, wherein the source of dianion is added to the aqueous solution comprising the chloride anion and manganese-containing coordination complex at least 30 seconds, at least 1 minute, at least 5 minutes, at least 10 minutes, at least 30 minutes, and/or at least one hour after combining the manganese-containing coordination complex with the source of chloride anion in the aqueous solution.

37. The method according to any preceding claim, further comprising bringing the aqueous solution to a pH of at least 8 prior to combining the manganese-containing coordination complex and source of chloride anion therein.

38. The method according to any preceding claim, wherein the aqueous formulation comprises a buffered formulation for parenteral administration of the manganese-containing coordination complex, the buffered formulation having a physiological level of sodium chloride.

39. The method according to any preceding claim, wherein a concentration of the manganese-containing coordination complex in the aqueous formulation is at least 2 mM, at least 6 mM, at least 18 mM, at least 20 mM, and/or at least 40 mM.

40. An aqueous formulation comprising a manganese-containing coordination complex, the aqueous formulation prepared according to a method corresponding to any preceding claim.

41. A method of treatment of a condition in a patient, comprising parenterally administering a buffered solution comprising the aqueous formulation of the manganese-containing coordination complex of any preceding claim.

42. The method of treatment according to claim 41, comprising intravenously administering the buffered solution comprising the manganese-containing coordination complex.

43. The method of treatment according to any one of claims 41 and 42, comprising parenterally administering the buffered solution comprising the manganese-containing coordinate complex to treat any selected from the group consisting of cancer, a cardiovascular disorder, a cerebrovascular disorder, a dermatological disorder, a fibrotic disorder, a gastrointestinal disorder, an immunological disorder, an inflammatory disorder, a metabolic disorder, a neurological disorder, an ophthalmic disorder, a pulmonary disorder, an infectious disease, and combinations thereof.

44. The method of treatment according to any of claims 41-43, comprising parenterally administering the buffered solution comprising the manganese-containing coordinate complex to treat cancer and/or a radiation-induced tissue injury.

45. A buffered formulation for parenteral administration of a manganese-containing pentaaza macrocyclic ring complex, the buffered formulation comprising:
a buffered aqueous solution comprising: (i) the manganese-containing pentaaza macrocyclic ring complex in a concentration of from 1 mg/mL to 50 mg/mL; (ii) sodium chloride in a concentration of from 130 mM to 160 mM; and (iii) a buffering agent comprising bicarbonate in a concentration sufficient to buffer the aqueous solution to a pH in the range of 7 to 10,
wherein a storage stability of the buffered formulation is such that no manganese-containing precipitate is detectable as measured via visual detection for 9 months following preparation of the buffered formulation.

46. The buffered formulation according to claim 45, wherein the visual detection storage stability of the buffered formulation is such that no manganese-containing precipitate is detectable as measured via visual detection after 1 day and/or after 6 days following formation of the buffered formulation.

47. The buffered formulation according to claim 45 or 46, wherein an ICP-MS storage stability assay comprises filtering the buffered formulation through a 0.45 micrometer filter, washing the filter with pH 8.0 water, digesting the filter contents with nitric acid, and performing inductively coupled mass-spectrometry (ICP-MS) to detect manganese content of any precipitate, and where the amount of manganese measured by the ICP-MS storage stability assay after at least 1 day, at least 6 days, and/or at least 9 months is less than 1500 ppm, and/or even less than 1200 ppm.

48. The buffered formulation according to any of claims 45-47, comprising bicarbonate in concertation of from 20 mM to 30 mM.

49. The buffered formulation according to any of claims 45-48, as formed via any of the methods of claims 1-39.
